(19) United States
(12) Patent Application Publication
(10) Pub. No.: US 2026/0349343 A1
(43) Pub. Date: Nov. 5, 2026

(54) RADIATION-SENSITIVE COMPOSITION AND PATTERN FORMING METHOD

(71) Applicant: JSR CORPORATION, Tokyo (JP)

(72) Inventors: Asuka HACHIYA, Tokyo (JP); Katsuaki NISHIKORI, Tokyo (JP); Takuya OMIYA, Tokyo (JP)

(73) Assignee: JSR CORPORATION, Tokyo (JP)

(21) Appl. No.: 19/690,611

(22) Filed: May 28, 2026

Related U.S. Application Data
(63) Continuation-in-part of application No. PCT/JP2024/044195, filed on Dec. 13, 2024.

(30) Foreign Application Priority Data
Jan. 18, 2024 (JP) 2024-006261

(51) Int. Cl. G03F 7/039; C08F 212/14; C08F 220/18; C08F 220/22; C08F 220/40; G03F 7/00; G03F 7/004; G03F 7/20; H10P 76/20

(52) U.S. Cl. CPC G03F 7/0397; C08F 212/22; C08F 220/1804; C08F 220/1807; C08F 220/1809; C08F 220/1811; C08F 220/1812; C08F 220/22; C08F 220/40; G03F 7/0046; C08F 2800/10; G03F 7/2004; G03F 7/70033; H10P 76/2041

(57) ABSTRACT

A radiation-sensitive composition includes: a polymer including: a structural unit (I) which includes an acid-dissociable group; and, optionally, a structural unit (II) which includes an acid generation structure that includes a first organic acid anion and an iodonium cation; a radiation-sensitive acid generator that includes a second organic acid anion and a second onium cation; an acid diffusion controlling agent that includes a third organic acid anion and a third onium cation. The radiation-sensitive acid generator includes a compound represented by formula (1). At least one of conditions (i) and (ii) is satisfied: (i) the polymer includes the structural unit (II), and (ii) at least a part of the second onium cation or the third onium cation is an iodonium cation. W is a (p+q+1)-valent aromatic ring having 5 to 20 ring members.

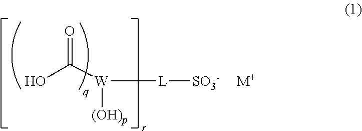

### CROSS-REFERENCE TO RELATED APPLICATIONS

The present application is a continuation-in-part application of International Patent Application No. PCT/JP2024/044195 filed Dec. 13, 2024, which claims priority to Japanese Patent Application No. 2024-006261 filed Jan. 18, 2024. The contents of these applications are incorporated herein by reference in their entirety.

### BACKGROUND OF THE DISCLOSURE

### Technical Field

The present disclosure relates to a radiation-sensitive composition and a pattern forming method.

### Background Art

A photolithography technique performed using a resist composition has been utilized for formation of a fine circuit on a semiconductor device. As the representative procedure, for example, a resist pattern is formed on a substrate by generating an acid by irradiating the coating of the resist composition with radioactive ray through a mask pattern, and then generate a difference in solubility of polymer into an alkaline or organic developer between an exposed part and a non-exposed part through a reaction in the presence of the acid as a catalyst.

In the photolithography technique, pattern miniaturization is promoted by using short-wavelength radiation such as ArF excimer laser, or by combining such radiation with an immersion exposure method (liquid immersion lithography). As a next generation technique, the use of further shorter-wavelength radiation such as an electron beam, X-ray, and extreme ultraviolet (EUV) is being sought.

To form a finer resist pattern in the formation of a circuit of a semiconductor device by a photolithography technique, various studies have been conducted on a photoacid generator, which is one of main components of a resist composition (see, for example, JP-A-2020-75910 and JP-B-5083528).

### SUMMARY

According to an aspect of the present disclosure, a radiation-sensitive composition includes: a polymer including: a structural unit (I) which includes an acid-dissociable group; and, optionally, a structural unit (II) which includes an acid generation structure that includes a first organic acid anion and an iodonium cation liberated from the first organic acid anion and is capable of generating through exposure an acid which dissociates the acid-dissociable group; a radiation-sensitive acid generator that includes a second organic acid anion and a second onium cation liberated from the second organic acid anion and is capable of generating through exposure an acid which dissociates the acid-dissociable group; an acid diffusion controlling agent that includes a third organic acid anion and a third onium cation liberated from the third organic acid anion and is capable of generating through exposure an acid which does not dissociate the acid-dissociable group; and a solvent. The radiation-sensitive acid generator includes a compound represented by formula (1). At least one of conditions (i) and (ii) is satisfied: (i) the polymer includes the structural unit (II), and (ii) at least a part of the second onium cation or the third onium cation is an iodonium cation.

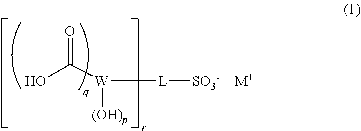

In the formula (1), W is a (p+q+1)-valent aromatic ring having 5 to 20 ring members, when there are a plurality of Ws, the plurality of Ws are identical to or different from each other, L is a (r+1)-valent linking group, r is an integer of 1 to 3, p is an integer of 0 to 3, when there are a plurality of ps, the plurality of ps are identical to or different from each other, q is an integer of 1 to 3, when there are a plurality of qs, the plurality of qs are identical to or different from each other, and M⁺ is a monovalent onium cation.

According to another aspect of the present disclosure, a pattern formation method includes: directly or indirectly applying the above-described radiation-sensitive composition onto a substrate to form a resist film; exposing the resist film to light; and developing the exposed resist film with a developer.

### DETAILED DESCRIPTION OF THE PREFERRED EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed.

Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

The inventors found that introducing iodonium cations, which decompose efficiently upon exposure, into components containing acid-generating structures could improve acid generation efficiency.

Based on this finding, the inventors have investigated the application of iodonium cations to enhance the sensitivity of resist compositions. However, the inventors' investigations revealed that resist compositions using iodonium cations exhibit reduced storage stability.

The radiation-sensitive composition can exhibit excellent sensitivity and CDU in resist pattern formation, and has good storage stability. The reason for this is not clear, but can be presumed as follows.

In the radiation-sensitive composition, since an iodonium cation having high decomposition performance through exposure to light is introduced into at least the acid generation structure contained in the structural unit (II) in the polymer, the radiation-sensitive acid generator, or the acid diffusion controlling agent, the acid generation efficiency can be increased, and as a result, high sensitivity can be achieved. In addition, introducing a carboxy group into the radiation-sensitive acid generator can increase the dissolution contrast by improving the solubility in an alkaline developer or the insolubility in an organic solvent developer, or can control the acid diffusion length by interaction with other components, so that good CDU can be exhibited. Furthermore, supplying an acid from the carboxy group of the radiation-sensitive acid generator can appropriately adjust the basicity of the acid diffusion controlling agent to suppress the decomposition of the iodonium cation. According to these comprehensive effects, it is presumed that the radiation-sensitive composition can exhibit various performances described above.

In the pattern formation method, since the radiation-sensitive composition capable of exhibiting excellent sensitivity and CDU in resist pattern formation, and having good storage stability is used, a high-quality resist pattern can be efficiently formed.

Hereinafter, embodiments of the present invention will be described in detail, but the present invention is not limited to these embodiments. Combinations of preferred embodiments are also preferable.

### <<Radiation-Sensitive Composition>>

The radiation-sensitive composition (hereinafter also simply referred to as “composition”) according to the present embodiment contains a polymer (hereinafter also referred to as “base polymer”), a radiation-sensitive acid generator, an acid diffusion controlling agent, and a solvent. In the composition, the iodonium cation is introduced into at least one of (the acid generation structure of) the base polymer, the radiation-sensitive acid generator, or the acid diffusion controlling agent. Examples of contained aspects of the iodonium cation in the composition include the following contained aspect (1) to contained aspect (7). Throughout the present specification, the numbers of these contained aspects are used.

(1) An aspect in which a base polymer into which an iodonium cation is introduced; a radiation-sensitive acid generator into which no iodonium cation is introduced; and an acid diffusion controlling agent into which no iodonium cation is introduced are included.

(2) An aspect in which a base polymer into which no iodonium cation is introduced; a radiation-sensitive acid generator into which an iodonium cation is introduced; and an acid diffusion controlling agent into which no iodonium cation is introduced are included.

(3) An aspect in which a base polymer into which no iodonium cation is introduced; a radiation-sensitive acid generator into which no iodonium cation is introduced; and an acid diffusion controlling agent into which an iodonium cation is introduced are included.

(4) An aspect in which a base polymer into which an iodonium cation is introduced; a radiation-sensitive acid generator into which no iodonium cation is introduced; and an acid diffusion controlling agent into which an iodonium cation is introduced are included.

(5) An aspect in which a base polymer into which an iodonium cation is introduced; a radiation-sensitive acid generator into which an iodonium cation is introduced; and an acid diffusion controlling agent into which no iodonium cation is introduced are included.

(6) An aspect in which a base polymer into which no iodonium cation is introduced; a radiation-sensitive acid generator into which an iodonium cation is introduced; and an acid diffusion controlling agent into which an iodonium cation is introduced are included.

(7) An aspect in which a base polymer into which an iodonium cation is introduced; a radiation-sensitive acid generator into which an iodonium cation is introduced; and an acid diffusion controlling agent into which an iodonium cation is introduced are included.

The composition may contain other optional components as long as the effects of the present invention are not impaired.

### <Polymer>

The polymer (that is, the base polymer) is an aggregate of polymerized chains containing the structural unit (I) having an acid-dissociable group. The base polymer may contain the structural unit (II) containing an acid generating structure, a structural unit having a phenolic hydroxy group (hereinafter, also referred to as “structural unit (III)”), and the like, in addition to the structural unit (I).

### (Structural Unit (I))

The structural unit (I) is a structural unit having an acid-dissociable group. The “acid-dissociable group” refers to a group that substitutes for a hydrogen atom of a carboxy group, a phenolic hydroxy group, an alcoholic hydroxy group, a sulfo group, or the like, and is dissociated by the action of an acid. The acid generated from the acid generation structure when the base polymer contains the structural unit (II) or the radiation-sensitive acid generator through exposure to light dissociates the acid-dissociable group in the structural unit (I) to generate a carboxy group or the like. As a result, a difference in solubility into a developer arises between the exposed portion and the unexposed portion of a resist film, making it possible to achieve pattern formation.

The structural unit (I) is not particularly limited as long as the structural unit (I) has an acid-dissociable group, and examples thereof include a structural unit having a tertiary alkyl ester moiety, a structural unit having a structure in which a hydrogen atom of a phenolic hydroxy group is replaced by a tertiary alkyl group, and a structural unit having an acetal linkage. A structural unit represented by formula (1) (hereinafter also referred to as “structural unit (I-1)”) is preferable from the viewpoint of improvement in the patternability of the radiation-sensitive composition.

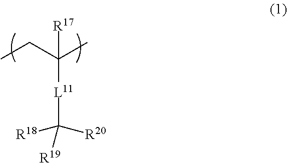

In the formula (1), R¹⁷ is a hydrogen atom, a fluorine atom, a methyl group, or a trifluoromethyl group. R¹⁸ is a substituted or unsubstituted monovalent hydrocarbon group having 1 to 20 carbon atoms. R¹⁹ and R²⁰ are each independently a substituted or unsubstituted monovalent chain hydrocarbon group having 1 to 10 carbon atoms or a substituted or unsubstituted monovalent alicyclic hydrocarbon group having 3 to 20 carbon atoms, or represent a divalent alicyclic group having 3 to 20 carbon atoms composed of R¹⁹ and R²⁰ combined with each other together with a carbon atom to which R¹⁹ and R²⁰ are bonded. L¹¹ represents *—COO—, *-L^{11a}-COO— or *—COO-L^{11a}-. L^{11a} is a substituted or unsubstituted alkanediyl group, or a substituted or unsubstituted arenediyl group. * is a bond to a carbon atom to which R¹⁷ is bonded.

As R¹⁷, a hydrogen atom and a methyl group are preferable from the viewpoint of the copolymerizability of a monomer that affords the structural unit (I-1), and a methyl group is more preferable.

Examples of the alkanediyl group represented by L^{11a} include divalent alkanediyl groups having 1 to 10 carbon atoms such as a methanediyl group, an ethanediyl group, and a propanediyl group. The alkanediyl group represented by L^{11a} is preferably a methanediyl group or an ethanediyl group.

Examples of the arenediyl group represented by L^{11a} include divalent aromatic hydrocarbon groups having 6 to 20 carbon atoms such as a benzenediyl group and a naphthalenediyl group. As the arenediyl group represented by L^{11a}, a benzenediyl group is preferable.

Examples of the substituent which the alkanediyl group or the arenediyl group represented by L^{11a} can have include halogen atoms (a fluorine atom, a chlorine atom, a bromine atom, an iodine atom, etc.), a carboxy group, a cyano group, a nitro group, a hydroxyl group, an alkyl group, a fluorinated alkyl group, an alkoxycarbonyl group, an alkoxycarbonyloxy group, an acyl group, an acyloxy group, and an alkoxy group.

Examples of the alkyl group as a substituent of L^{11a} include linear or branched alkyl groups having 1 to 8 carbon atoms such as a methyl group, an ethyl group, and a propyl group. Examples of the fluorinated alkyl group include linear or branched fluorinated alkyl groups having 1 to 8 carbon atoms such as a trifluoromethyl group and a pentafluoroethyl group. Examples of alkoxycarbonyl groups include alkoxycarbonyl groups having 1 to 6 carbon atoms, such as methoxycarbonyl groups and ethoxycarbonyl groups. Examples of the alkoxycarbonyloxy group include chain or alicyclic alkoxycarbonyloxy groups having 2 to 16 carbon atoms such as a methoxycarbonyloxy group, a butoxycarbonyloxy group, and an adamantylmethyloxycarbonyloxy group. Examples of the acyl group include aliphatic or aromatic acyl groups having 2 to 12 carbon atoms such as an acetyl group, a propionyl group, a benzoyl group, and an acryloyl group. Examples of the acyloxy group include aliphatic or aromatic acyloxy groups having 2 to 12 carbon atoms such as an acetyloxy group, a propionyloxy group, a benzoyloxy group, and an acryloyloxy group. Examples of the alkoxy group include linear or branched alkoxy groups having 1 to 8 carbon atoms such as a methoxy group, an ethoxy group, and a propoxy group.

Examples of the monovalent hydrocarbon group having 1 to 20 carbon atoms represented by R¹⁸ include a monovalent chain hydrocarbon group having 1 to 20 carbon atoms, a monovalent alicyclic hydrocarbon group having 3 to 20 carbon atoms, and a monovalent aromatic hydrocarbon group having 6 to 20 carbon atoms.

Examples of the monovalent chain hydrocarbon group having 1 to 20 carbon atoms include: linear or branched alkyl groups such as a methyl group, an ethyl group, an n-propyl group, an isopropyl group, an n-butyl group, a sec-butyl group, an isobutyl group, a tert-butyl group, an n-pentyl group, and an isopentyl group; alkenyl groups such as an ethenyl group, a propenyl group, and a butenyl group; and alkynyl groups such as an ethynyl group, a propynyl group, and a butynyl group.

Examples of the monovalent alicyclic hydrocarbon group having 3 to 20 carbon atoms include monocyclic alicyclic saturated hydrocarbon groups such as a cyclopentyl group, a cyclohexyl group, a cycloheptyl group, and a cyclooctyl group; polycyclic alicyclic saturated hydrocarbon groups such as a norbornyl group, an adamantyl group, a tricyclodecyl group, and a tetracyclododecyl group; monocyclic alicyclic unsaturated hydrocarbon groups such as a cyclopentenyl group and a cyclohexenyl group; and polycyclic alicyclic unsaturated hydrocarbon groups such as a norbornenyl group, a tricyclodecenyl group, and a tetracyclododecenyl group.

Examples of the monovalent aromatic hydrocarbon group having 6 to 20 carbon atoms include aryl groups such as a phenyl group, a tolyl group, a xylyl group, a naphthyl group, and an anthryl group; and aralkyl groups such as a benzyl group, a phenethyl group, a naphthylmethyl group, and an anthrylmethyl group.

R¹⁸ is preferably a linear or branched saturated hydrocarbon group having 1 to 10 carbon atoms or an aromatic hydrocarbon group having 6 to 20 carbon atoms.

Examples of the monovalent chain hydrocarbon group having 1 to 10 carbon atoms represented by R¹⁹ and R²⁰ include groups corresponding to 1 to 10 carbon atoms among the monovalent chain hydrocarbon groups having 1 to 20 carbon atoms as R¹⁸.

Examples of the monovalent alicyclic hydrocarbon group having 3 to 20 carbon atoms represented by R¹⁹ and R²⁰ include the monovalent alicyclic hydrocarbon groups having 3 to 20 carbon atoms as R¹⁸.

The divalent alicyclic group having 3 to 20 carbon atoms composed of R¹⁹ and R²⁰ combined with each other together with the carbon atom to which R¹⁹ and R²⁰ are bonded is not particularly limited as long as the divalent alicyclic group is a group obtained by removing two hydrogen atoms from an identical carbon atom constituting a carbon ring of a monocyclic or polycyclic alicyclic hydrocarbon having the aforementioned number of carbon atoms. The divalent alicyclic group may be either a monocyclic hydrocarbon group or a polycyclic hydrocarbon group, and the polycyclic hydrocarbon group may be either a bridged alicyclic hydrocarbon group or a fused alicyclic hydrocarbon group, and may be either a saturated hydrocarbon group or an unsaturated hydrocarbon group.

Among the monocyclic alicyclic hydrocarbon groups, a cyclopentanediyl group, a cyclohexanediyl group, a cycloheptanediyl group, a cyclooctanediyl group, and the like are preferable as saturated hydrocarbon groups, and a cyclopentenediyl group, a cyclohexenediyl group, a cycloheptenediyl group, a cyclooctenediyl group, a cyclodecenediyl group, and the like are preferable as unsaturated hydrocarbon groups. As the polycyclic alicyclic hydrocarbon group, bridged alicyclic saturated hydrocarbon groups are preferable, and for example, a bicyclo[2.2.1]heptane-2,2-diyl group (norbornane-2,2-diyl group), a bicyclo[2.2.2]octane-2,2-diyl group, and a tricyclo[3.3.1.1^{3,7}]decane-2,2-diyl group (adamantane-2,2-diyl group) are preferable.

Among them, it is preferable that R¹⁸ is an alkyl group having 1 to 4 carbon atoms, an alkenyl group, or a substituted or unsubstituted phenyl group, and the alicyclic structure constituted by R¹⁹ and R²⁰ combined with each other and the carbon atom to which the R¹⁹ and R²⁰ are bonded is a polycyclic or monocyclic cycloalkane structure.

As the substituent which R¹⁸ to R²⁰ can have, a substituent which the alkanediyl group or the arenediyl group represented by L^{11a} can have can be suitably employed.

Examples of the structural unit (I-1) include structural units represented by formulas (3-1) to (3-11) (hereinafter also referred to as “structural units (I-1-1) to (I-1-11)”).

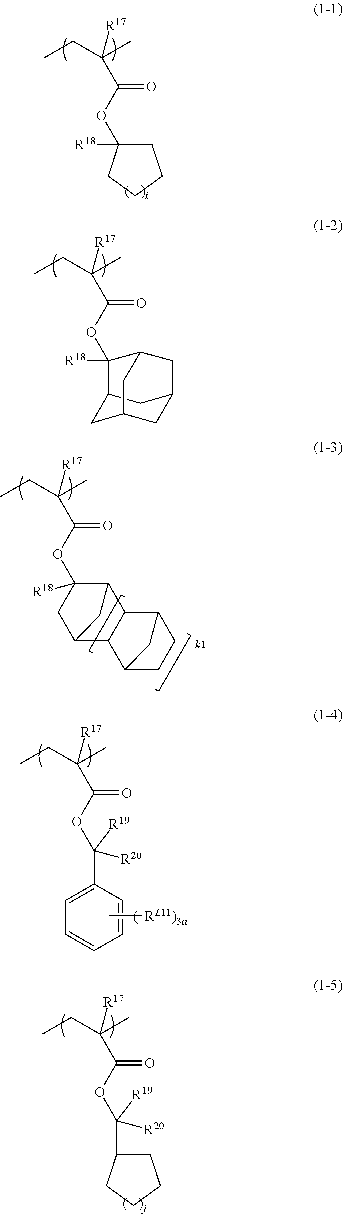

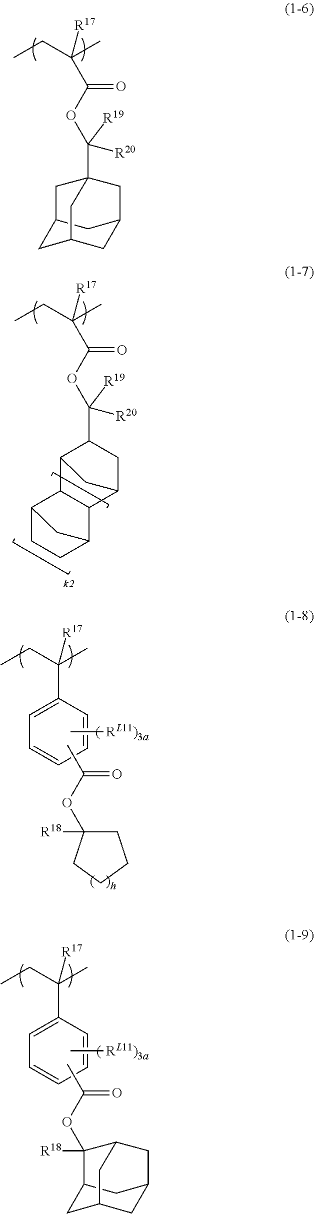

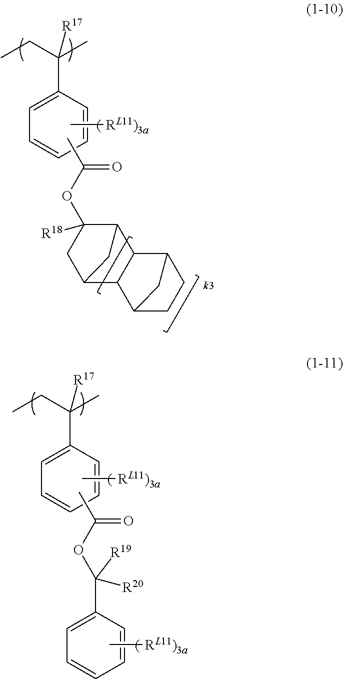

In the above formulas (3-1) to (3-11), R¹⁷ to R²⁰ have the same meaning as in the above formula (1). R^{L11} is a halogen atom, a carboxy group, a cyano group, a nitro group, a hydroxy group, an alkyl group, a fluorinated alkyl group, an alkoxycarbonyl group, an alkoxycarbonyloxy group, an acyl group, an acyloxy group, or an alkoxy group. h, i and j are each independently an integer of 1 to 4. K1, k2 and k3 are each independently 0 or 1. 3a is independently at each occurrence an integer of 0 to 3. When 3a is 2 or more, the plurality of R^{L11}s are the same or different from each other.

As h, i and j, 1 or 2 is preferable. As R¹⁸, a methyl group, an ethyl group, an isopropyl group, t-butyl group, an ethenyl group (vinyl group), a phenyl group, and an iodophenyl group are preferable. As R¹⁹ and R²⁰, a methyl group, an ethyl group, and an isopropyl group are preferable. As R^{L11}, an iodine atom, an alkyl group and an alkoxy group are preferable. It is preferable to introduce an iodo group into the structural unit (I) by employing an iodophenyl group as R¹⁸ or employing an iodo atom as R^{L11}. By the introduction of an iodo group, the radiation absorption efficiency is increased, and the secondary electron-generating efficiency is increased, so that the sensitivity can be improved.

Furthermore, the polymer may contain structural units represented by formulas (1f) to (2f) as the structural unit (I).

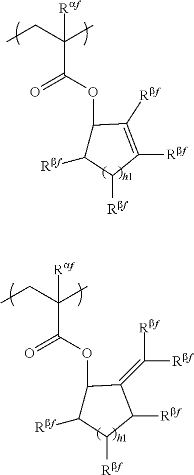

In the formulas (1f) to (2f), R^{αf}s are each independently a hydrogen atom, a fluorine atom, a methyl group, or a trifluoromethyl group. Refs are each independently a hydrogen atom or a chain alkyl group having 1 to 5 carbon atoms. h1 is an integer of 1 to 4.

As the R^{βf}, a hydrogen atom, a methyl group, or an ethyl group is preferable. h1 is preferably 1 or 2.

Specific examples of the structural unit (I) (including the structural unit (1-1)) include, but are not particularly limited to, structures represented by formulas (1-1) to (1-34).

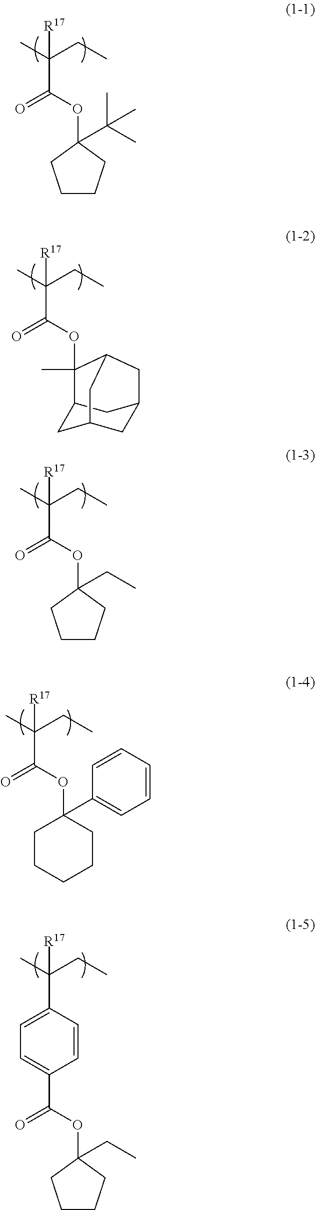

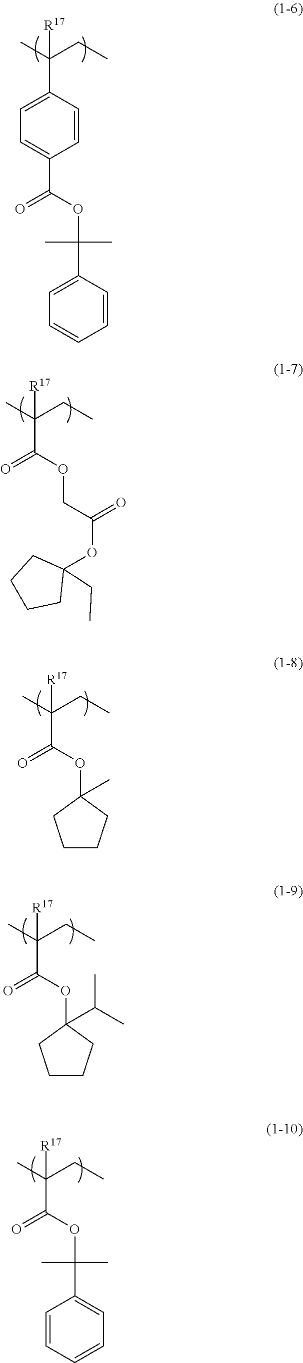

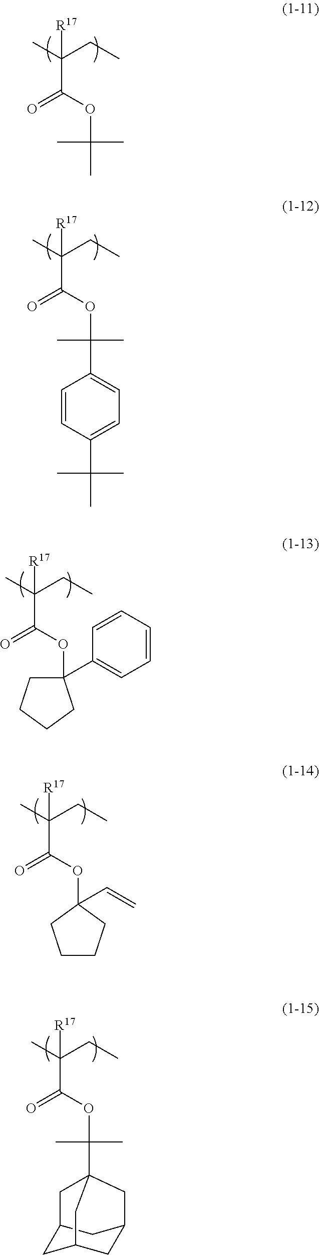

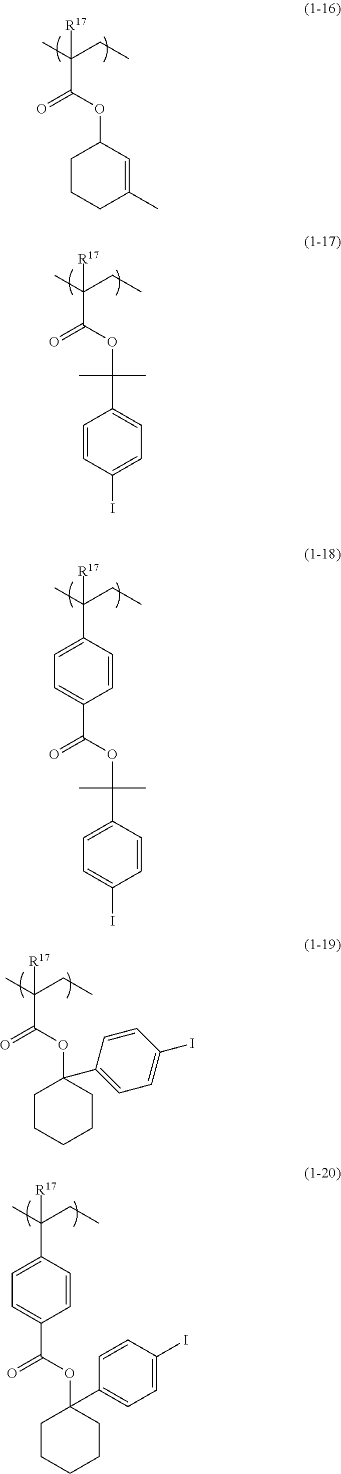

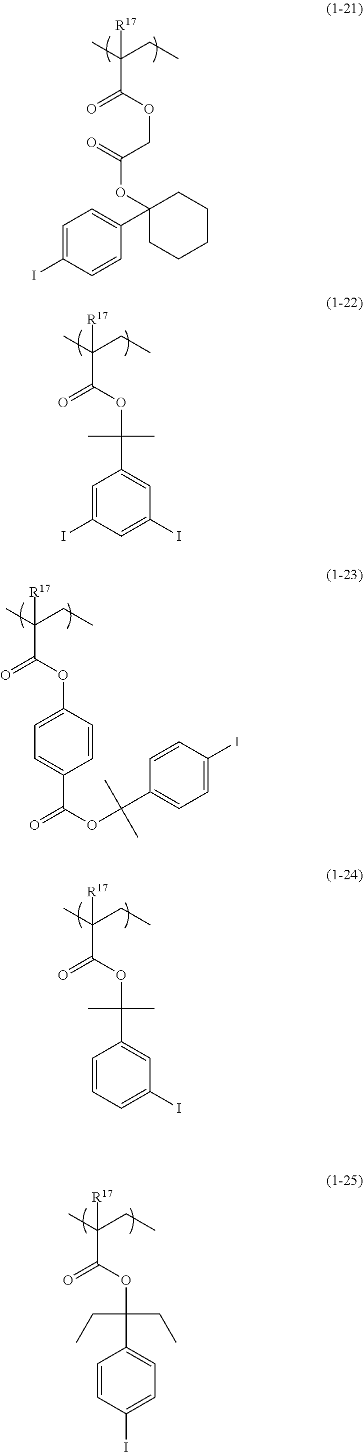

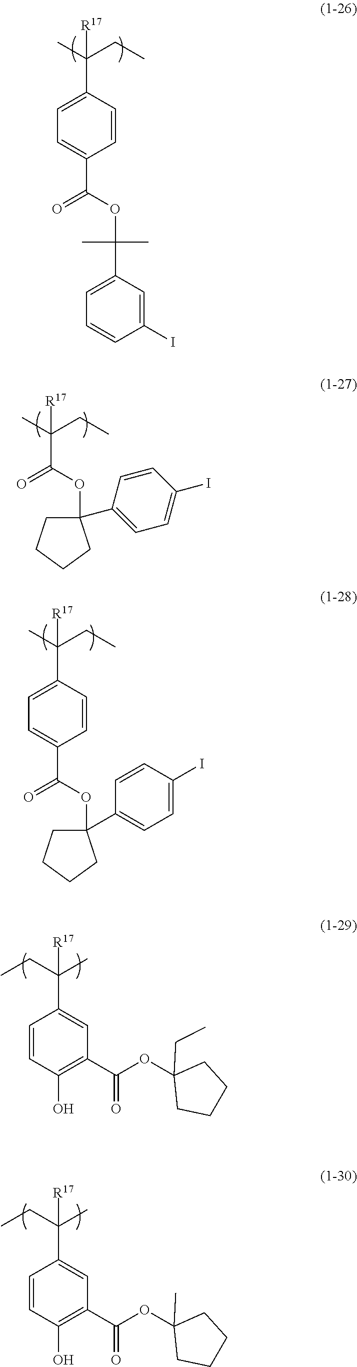

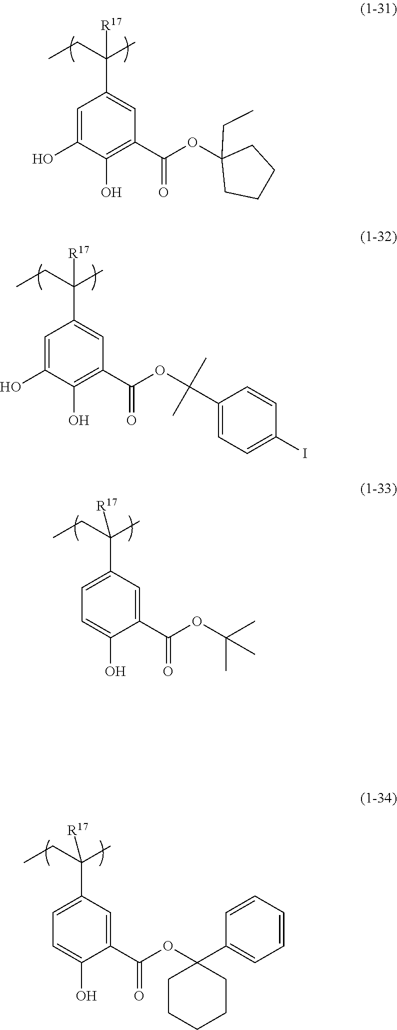

In the formulas, R¹⁷ has the same definition as in the formula (1).

The lower limit of the content ratio of the structural unit (I) (the total content ratio when a plurality of structural units (I) is contained) is preferably 10 mol %, more preferably 20 mol %, and still more preferably 30 mol % based on all structural units constituting the base polymer. The upper limit of the content ratio is preferably 95 mol %, more preferably 90 mol %, and still more preferably 85 mol %. When the content ratio of the structural unit (I) is within the above range, the patternability of the radiation-sensitive composition can be further improved. When the acid-dissociable group has the iodo group, sensitivity can be further improved.

### (Structural Unit (II))

The structural unit (II) includes an acid generating structure. The acid generating structure has a first organic acid anion and a first onium cation and generates an acid that dissociates the acid-dissociable group through exposure to light. The onium salt structure formed of the first organic acid anion and the first onium cation (that is, the acid generating structure) functions as a so-called radiation-sensitive acid generating structure.

In the case of the contained aspects (1), (4), (5) and (7), the first onium cation is an iodonium cation liberated from the first organic acid anion. In the case of aspects other than these aspects, the first onium cation is another cation.

In the specification, the “dissociation” of the acid-dissociable group refers to dissociation that occurs when post-exposure baking is performed at 100 °C for 60 seconds.

When the base polymer contains the radiation-sensitive acid generating structure, the polarity of the base polymer of the exposed portion increases, and therefore when the developer is an aqueous alkaline solution, the base polymer is soluble, and on the other hand, when the developer is an organic solvent, the base polymer is hardly soluble in the developer.

The form of the first organic acid anion and the first onium cation contained in the structural unit (II) of the base polymer is not particularly limited, and the base polymer may have the first organic acid anion as a side chain portion or may have the first onium cation as a side chain portion. Having as a side chain portion means that the corresponding first organic acid anion or first onium cation is bonded (covalently bonded) to the main chain as a side chain structure of the base polymer. When the first organic acid anion is bonded to the main chain as a side chain structure of the base polymer, the first onium cation is ionically bonded to the first organic acid anion as a counter ion of the first organic acid anion. On the other hand, when the first onium cation is bonded to the main chain as a side chain structure of the base polymer, the first organic acid anion is ionically bonded to the first onium cation as a counter ion of the first onium cation. From the viewpoint of controlling the acid diffusion length, the base polymer preferably has the first organic acid anion as a side chain portion.

The first organic acid anion preferably has at least one anion selected from the group consisting of a sulfonate anion, a carboxylate anion, and a sulfonimide anion as an acid anion moiety. Examples of the acid generated through exposure to light may include a sulfonic acid, a carboxylic acid, and a sulfonimide corresponding to the acid anion moiety.

The first organic acid anion preferably includes —O—, —CO—, a cyclic structure, or a combination thereof as a structure other than the acid anion moiety.

The cyclic structure may be any of a monocyclic structure, a polycyclic structure, or a combination thereof. The cyclic structure may be any of an alicyclic structure, an aromatic ring structure, a heterocyclic structure, or a combination thereof. In the case of the combination, the ring structure may be a structure in which ring structures are bonded by a chain structure, or two or more ring structures may form a fused ring structure, a bridged ring structure, or a spiro ring structure.

A divalent heteroatom-containing group may be present between carbons forming the skeleton of the cyclic structure or the chain structure, and some or all of hydrogen atoms on carbon atoms of the cyclic structure or the chain structure may be replaced by another substituent.

As the alicyclic structure, a structure corresponding to the monovalent alicyclic hydrocarbon group having 3 to 20 carbon atoms in R¹⁸ of the formula (1) can be suitably employed.

Examples of the aromatic ring structure include aromatic hydrocarbon rings such as a benzene ring, a naphthalene ring, an anthracene ring, a phenalene ring, a phenanthrene ring, a pyrene ring, a fluorene ring, a perylene ring, and a coronene ring. Among them, a benzene ring is preferable as the aromatic ring.

Examples of the heterocyclic structure include:
oxygen atom-containing aliphatic heterocyclic structures such as oxirane, tetrahydrofuran, tetrahydropyran, dioxolane, and dioxane;
nitrogen atom-containing aliphatic heterocyclic structures such as aziridine, pyrrolidine, piperidine, and piperazine;
sulfur atom-containing aliphatic heterocyclic structures such as thietane, thiolane, and thiane;
aliphatic heterocyclic structures containing multiple types of heteroatoms such as morpholine, 1,2-oxathiolane, and 1,3-oxathiolane;
oxygen atom-containing aromatic heterocyclic structures such as furan and benzofuran;
nitrogen atom-containing aromatic heterocyclic structures such as pyrrole, pyrazole, and triazine;
sulfur atom-containing aromatic heterocyclic structures such as thiophene; and
aromatic heterocyclic structures containing multiple types of heteroatoms such as oxazole, isothiazole, and thiazine.

The heterocyclic structure includes a lactone structure, a cyclic carbonate structure, a sultone structure, a cyclic acetal, or a combination thereof. Examples of such structures include structures represented by formulas (H-1) to (H-11).

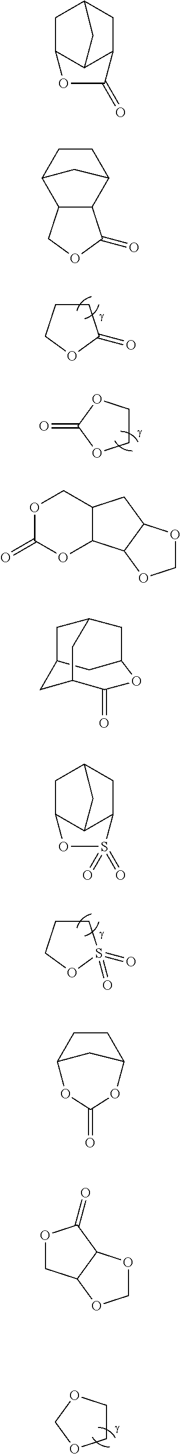

In the formulas, γ is an integer of 1 to 3.

As the chain structure, structures corresponding to the monovalent chain hydrocarbon groups having 1 to 20 carbon atoms in R¹⁸ in the formula (1) can be suitably employed.

Examples of the divalent heteroatom-containing group include —CO—, —CS—, —NR′—, —O—, —S—, —SO₂—, or a divalent group obtained by combining them. R′ is a hydrogen atom or a hydrocarbon group having 1 to 10 carbon atoms.

As the substituent with which some or all of the hydrogen atoms on the carbon atoms of the cyclic structure or the chain structure are substituted, a substituent that can be possessed by the substituent which the alkanediyl group or the arenediyl group represented by L^{11a} in the formula (1) can have can be suitably employed.

In the first acid generating structure, it is preferable that the first organic acid anion have a sulfonate anion as an acid anion moiety, and an electron attractive group be bonded to a carbon atom adjacent to a sulfur atom in the sulfonate anion. As a result, the first acid generating structure can efficiently exhibit the above function. Examples of the electron attractive group include a fluorine atom, a fluorinated hydrocarbon group, a nitro group, and a cyano group. As the fluorinated hydrocarbon group, a perfluoroalkyl group having 1 to 5 carbon atoms is preferable.

The first organic acid anion preferably has an iodo group. The first organic acid anion preferably contains the iodo group-containing aromatic ring structure as an aspect of containing an iodo group. The iodo group-containing aromatic ring structure is a structure in which some or all of hydrogen atoms of the aromatic ring are replaced by an iodo group. Examples of the aromatic ring in the iodo group-containing aromatic ring structure include aromatic hydrocarbon rings such as a benzene ring, a tolyl ring, a naphthalene ring, an anthracene ring, a phenalene ring, a phenanthrene ring, a pyrene ring, a fluorene ring, a perylene ring, and a coronene ring; heteroaromatic rings such as a furan ring, a pyrrole ring, a thiophene ring, a phosphole ring, a pyrazole ring, an oxazole ring, an isoxazole ring, a thiazole ring, a pyridine ring, a pyrazine ring, a pyrimidine ring, a pyridazine ring, a triazine ring, a carbazole ring, and a dibenzofuran ring, or combinations thereof. Among them, a benzene ring is preferable as the aromatic ring. The number of iodo groups in the iodo group-containing aromatic ring structure is preferably 1, 2, or 3.

As described above, the first onium cation can be an iodonium cation or another cation depending on whether or not the iodonium cation is introduced into the radiation-sensitive acid generator, and whether or not the iodonium cation is introduced into the acid diffusion controlling agent. Examples of another cation include a sulfonium cation and a tetrahydrothiophenium cation. Among them, a sulfonium cation is preferable as another cation.

The first onium cation preferably has the iodo group. The first onium cation preferably contains the iodo group-containing aromatic ring structure as the mode of containing the iodo group.

The first onium cation in the structural unit (II) may be a fluoro group-containing onium cation having a fluoro group. The fluoro group-containing onium cation preferably has a fluoro group-containing aromatic ring structure. The fluoro group-containing aromatic ring structure is a structure in which some or all of hydrogen atoms of the aromatic ring are substituted with a fluoro group. As the aromatic ring in the fluoro group-containing aromatic ring structure, the aromatic ring in the iodo group-containing aromatic ring structure can be suitably employed. Thereby, the radiation absorption efficiency is increased, so that the sensitivity can be improved.

The first onium cation preferably contains at least one selected from the group consisting of an iodo group-containing aromatic ring structure and a fluoro group-containing aromatic ring structure.

As the structural unit (II) has the structures in combination, the functions described above can be efficiently exhibited.

The structural unit (II) is preferably a structural unit represented by formula (a1) (hereinafter, also referred to as “structural unit (II-1)”).

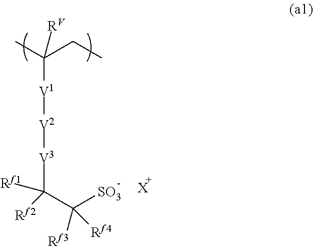

In the formulas, RV is a hydrogen atom or a methyl group. V¹ is a single bond or an ester group. V² is a linear, branched or cyclic alkylene group having 1 to 12 carbon atoms, a cycloalkylene group having 3 to 12 carbon atoms, an arylene group having 6 to 10 carbon atoms, a combination thereof, and a part of a methylene group composing the alkylene group, the cycloalkylene group, or the arylene group may be substituted with an ether group, an ester group, or a lactone ring-containing group. V³ is a single bond, an ether group, an ester group, a linear or branched alkylene group having 1 to 12 carbon atoms, or a cyclic cycloalkylene group having 3 to 12 carbon atoms, and a part of a methylene group composing the alkylene group may be substituted with an ether group or an ester group. Some or all of hydrogen atoms of V² and V³ may be substituted with a heteroatom or a monovalent hydrocarbon group having 1 to 20 carbon atoms optionally containing a heteroatom. Rf¹ to Rf⁴ are each independently a hydrogen atom, a fluorine atom, or a trifluoromethyl group, and at least one of Rf¹ to Rf⁴ is a fluorine atom or a fluorinated hydrocarbon group.

X⁺ is a monovalent iodonium cation or sulfonium cation. Details thereof will be described later.

The structural unit (II-1) is preferably represented by formula (a1-1).

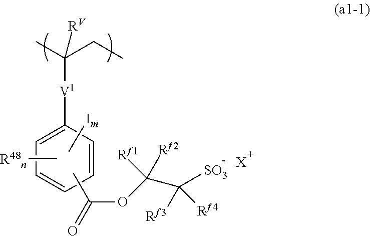

In the formulas, R^{V}, Rf¹ to Rf⁴, V¹ and X⁺ have the same meanings as in the formula (a1). R⁴⁸ is a linear, branched or cyclic alkyl group having 1 to 4 carbon atoms, a halogen atom other than iodine, a hydroxy group, a linear, branched or cyclic alkoxy group having 1 to 4 carbon atoms, or a linear, branched or cyclic alkoxycarbonyl group having 2 to 5 carbon atoms. m is an integer of 0 to 4. n is an integer of 0 to 3.

Examples of the first organic acid anion of the monomer that affords the structural unit (II) (including the structural unit (II-1)) include, but are not limited to, those shown below. The iodo group in the structure shown below may be substituted with a hydrogen atom, another substituent, or the like. In formulas, RV has the same meaning as described above.

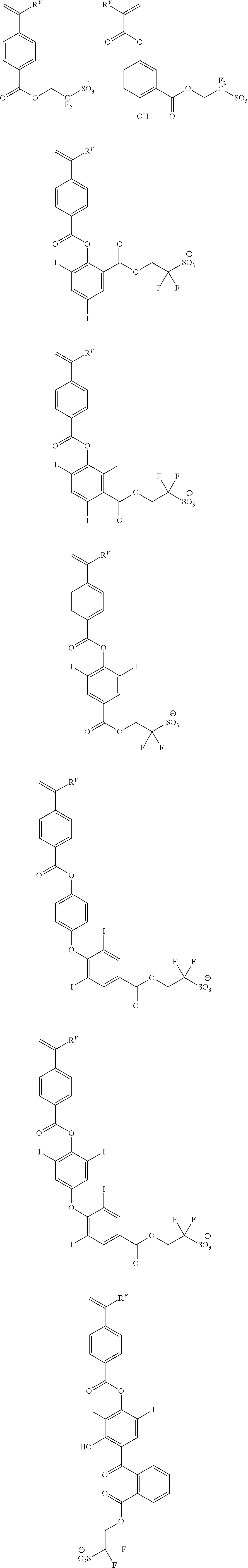

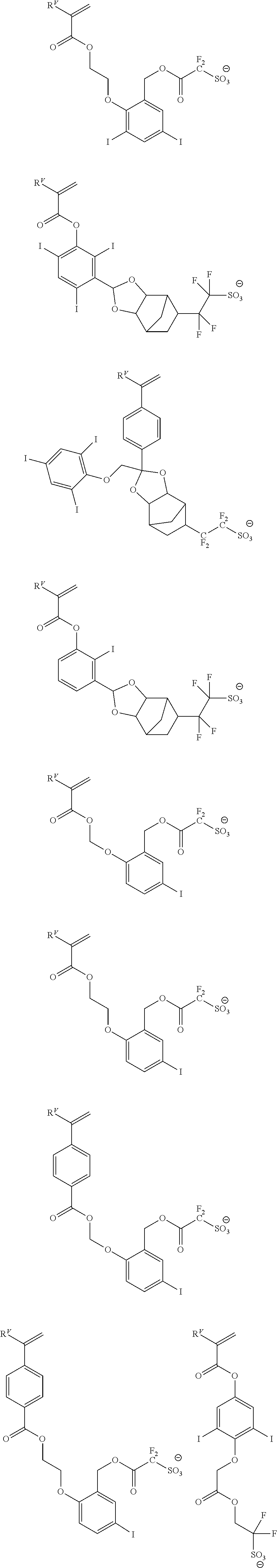

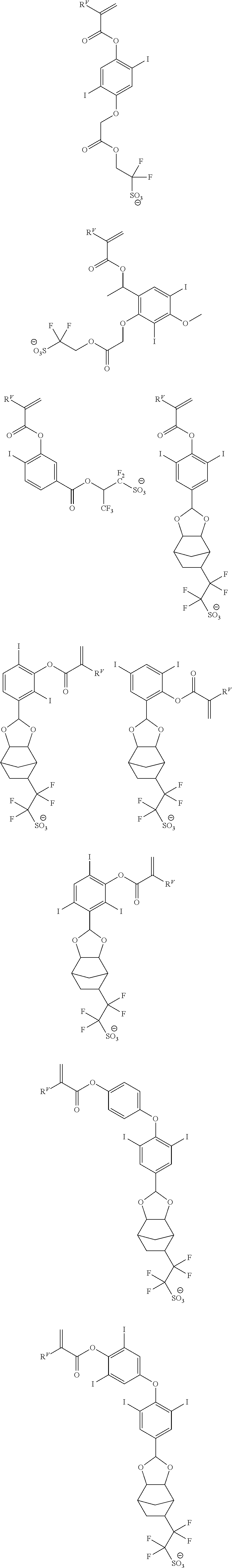

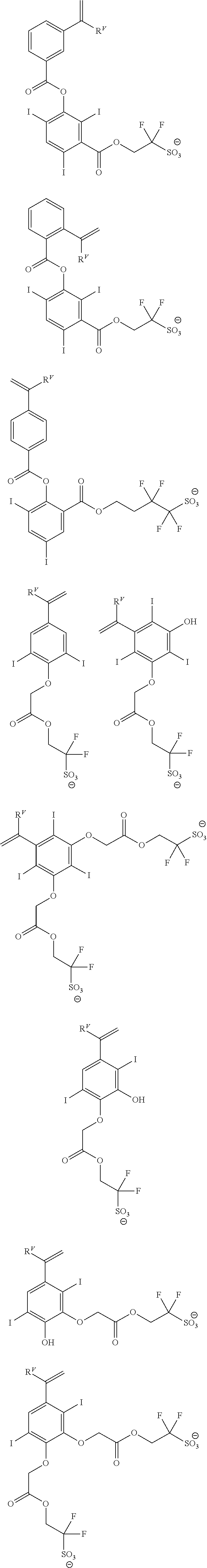

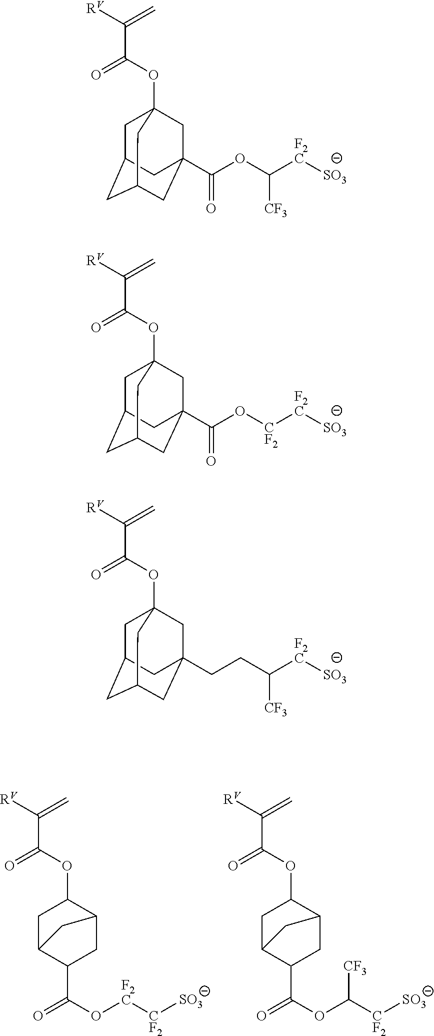

In the above formulas, RV has the same meaning as in the formula (a1).

The iodonium cation as the first onium cation of the structural unit (II-1) is preferably represented by formula (P1).

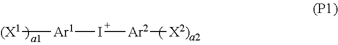

wherein
Ar¹ is an (a1+1)-valent aromatic ring having 5 to 20 carbon atoms,
Ar² is an (a2+1)-valent aromatic ring having 5 to 20 carbon atoms,
X¹ and X² are each independently a monovalent organic group having 1 to 20 carbon atoms, a nitro group, a hydroxy group, or a halogen atom, when a plurality of X¹s and X²s are present, the plurality of X¹s and X²s are identical to or different from each other, and
a1 and a2 are each independently an integer of 0 to 5.

In the present description, the “organic group” is a group containing at least one carbon atom.

As the aromatic ring in Ar¹ and Ar², among the aromatic rings in the iodo group-containing aromatic ring structure, a ring corresponding to 5 to 20 carbon atoms can be suitably employed. The (a1+1)-valent aromatic ring having 5 to 20 carbon atoms represented by Ar¹ has a structure obtained by removing (a1+1) hydrogen atoms from the aromatic ring. The (a2+1)-valent aromatic ring having 5 to 20 carbon atoms represented by Ar² has a structure obtained by removing (a2+1) hydrogen atoms from the aromatic ring. Ar¹ and Ar² are preferably benzene rings.

Examples of the monovalent organic groups having 1 to 20 carbon atoms represented by X¹ and X² include a monovalent hydrocarbon group having 1 to 20 carbon atoms, a group having a divalent heteroatom-containing group between carbon atoms or at the carbon chain terminal of the hydrocarbon group, a group obtained by replacing some or all of hydrogen atoms of the hydrocarbon group by a monovalent heteroatom-containing group, or a combination thereof. In addition, examples of the organic group include a cyano group and a carboxy group.

As the monovalent hydrocarbon group having 1 to 20 carbon atoms in the organic group, the monovalent hydrocarbon group having 1 to 20 carbon atoms represented by R¹⁸ of the formula (1) can be suitably employed. As the divalent heteroatom-containing group, the divalent heteroatom-containing group in the first organic acid anion of the structural unit (II) of the base polymer can be suitably employed. Examples of the monovalent heteroatom-containing group include a hydroxy group, a carboxy group, a sulfanyl group, a cyano group, a nitro group, and halogen atoms.

When X¹ and X² are present, X¹ and X² are each independently preferably an alkoxycarbonyl group or a halogen atom, and more preferably a fluorine atom or an iodine atom.

a1 and a2 each independently preferably represent an integer of 0 to 3, more preferably an integer of 0 to 2, and still more preferably 0 or 1.

Specific examples of such an iodonium cation represented by the formula (P1) include those shown below.

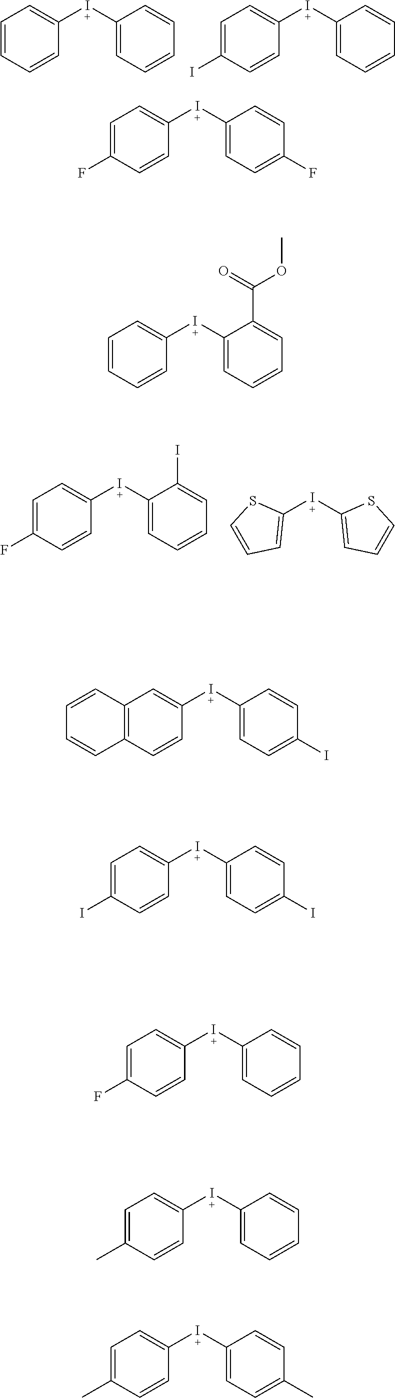

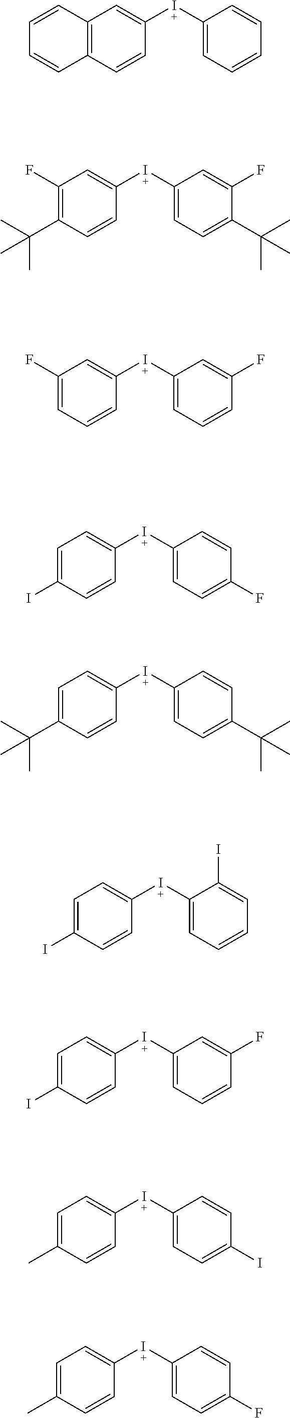

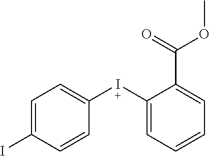

The sulfonium cation as the first onium cation of the structural unit (II-1) is preferably represented by formula (Q-1).

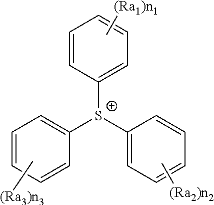

In the formula (Q-1), Ra1 and Ra2 each independently represent a substituent. n1 represents an integer of 0 to 5, and when n1 is 2 or more, the plurality of Ra1's may be the same as or different from each other. n2 represents an integer of 0 to 5, and when n2 is 2 or more, the plurality of Ra2's may be the same as or different from each other. n3 represents an integer of 0 to 5, and when n3 is 2 or more, the plurality of Ra3's may be the same as or different from each other. Ra3 represents a substituent. Ra1 and Ra2 may be linked to each other to form a ring. When n1 is 2 or more, the plurality of Ra1's may be linked to each other to form a ring. When n2 is 2 or more, the plurality of Ra2's may be linked to each other to form a ring.

As the substituents represented by Ra1, Ra2 and Ra3, an alkyl group, a cycloalkyl group, an alkoxy group, a cycloalkyloxy group, an alkoxycarbonyl group, an alkylsulfonyl group, a hydroxy group, a halogen atom, and a halogenated hydrocarbon group are preferable.

Examples of the alkyl groups of Ra1 and Ra2 include a linear or branched alkyl group shown as a monovalent chain hydrocarbon group having 1 to 20 carbon atoms in R¹⁸ of the formula (1). Among them, a methyl group, an ethyl group, an n-butyl group, and a t-butyl group are particularly preferable.

Examples of the cycloalkyl groups of Ra1 and Ra2 include monovalent alicyclic hydrocarbon groups having 3 to 20 carbon atoms in R¹⁸ of the formula (1). Among them, a cyclopropyl group, a cyclopentyl group, a cyclohexyl group, a cycloheptyl group, and a cyclooctyl group are particularly preferable.

Examples of the alkyl group moiety of the alkoxy group as Ra1 and Ra2 include those listed above as the alkyl group as Ra1 and Ra2. As the alkoxy group, a methoxy group, an ethoxy group, an n-propoxy group, and an n-butoxy group are particularly preferable.

Examples of the cycloalkyl group moiety of the cycloalkyloxy group as Ra1 and Ra2 include those listed above as the cycloalkyl group as Ra1 and Ra2. As the cycloalkyloxy group, a cyclopentyloxy group and a cyclohexyloxy group are particularly preferable.

Examples of the alkoxy group moiety of the alkoxycarbonyl group as Ra1 and Ra2 include those listed above as the alkoxy group as Ra1 and Ra2. As the alkoxycarbonyl group, a methoxycarbonyl group, an ethoxycarbonyl group, and an n-butoxycarbonyl group are particularly preferable.

Examples of the alkyl group moiety of the alkylsulfonyl group as Ra1 and Ra2 include those listed above as the alkyl group as Ra1 and Ra2. Examples of the cycloalkyl group moiety of the cycloalkylsulfonyl group as Ra1 and Ra2 include those listed above as the cycloalkyl group as Ra1 and Ra2. As the alkylsulfonyl group or the cycloalkylsulfonyl group, a methanesulfonyl group, an ethanesulfonyl group, an n-propanesulfonyl group, an n-butanesulfonyl group, a cyclopentanesulfonyl group, and a cyclohexanesulfonyl group are particularly preferable.

Each of the groups Ra1 and Ra2 may further have a substituent. Examples of the substituent include substituents that can be possessed by the alkanediyl group or the arenediyl group represented by L^{11a} of the formula (1).

Examples of the halogen atom as Ra1 and Ra2 include a fluorine atom, a chlorine atom, a bromine atom, and an iodine atom, and a fluorine atom and an iodine atom are preferable.

As the halogenated hydrocarbon group as Ra1 and Ra2, a halogenated alkyl group is preferable. Examples of the alkyl group and the halogen atom composing the halogenated alkyl group include those described above. Among them, a fluorinated alkyl group is preferable, and CF₃ is more preferable.

As described above, Ra1 and Ra2 may be linked to each other to form a ring (namely, a heterocyclic ring containing a sulfur atom). In this case, it is preferable that Ra1 and Ra2 be bonded to each other to form a single bond or a divalent linking group. Examples of the divalent linking group include —COO—, —OCO—, —CO—, —O—, —S—, —SO—, —SO₂—, an alkylene group, a cycloalkylene group, an alkenylene group, and combinations of two or more thereof, and those having 20 or less carbon atoms in total are preferable. When Ra1 and Ra2 are linked to each other to form a ring, it is preferable that Ra1 and Ra2 be bonded to each other to form-COO—, —OCO—, —CO—, —O—, —S—, —SO—, —SO₂—, or a single bond. Among them, it is more preferable to form —O—, —S—, or a single bond, and it is particularly preferable to form a single bond. When n1 is 2 or more, the plurality of Ra1's may be linked to each other to form a ring, and when n2 is 2 or more, the plurality of Ra2's may be linked to each other to form a ring. Examples thereof include an aspect in which two Ra1's are linked to each other to form a naphthalene ring together with a benzene ring to which two Ra1's are bonded.

Ra3 is preferably a fluorine atom, an iodine atom or a group having one or more fluorine atoms. Examples of the group having a fluorine atom may include groups in which the alkyl group, the cycloalkyl group, the alkoxy group, the cycloalkyloxy group, the alkoxycarbonyl group, and the alkylsulfonyl group as Ra1 and Ra2 are each substituted with a fluorine atom. Among them, fluorinated alkyl groups are suitable, CF₃ is more suitable.

Ra3 is preferably a fluorine atom or CF₃, and more preferably a fluorine atom.

n1 and n2 are each independently preferably an integer of 0 to 3, and more preferably an integer of 0 to 2.

n3 is preferably an integer of 1 to 3, and more preferably 1 or 2.

Examples of such an onium cation represented by the above formula (Q-1) include those shown below. The fluorine atom or the iodine atom in the following onium cations may be replaced by a hydrogen atom or another substituent.

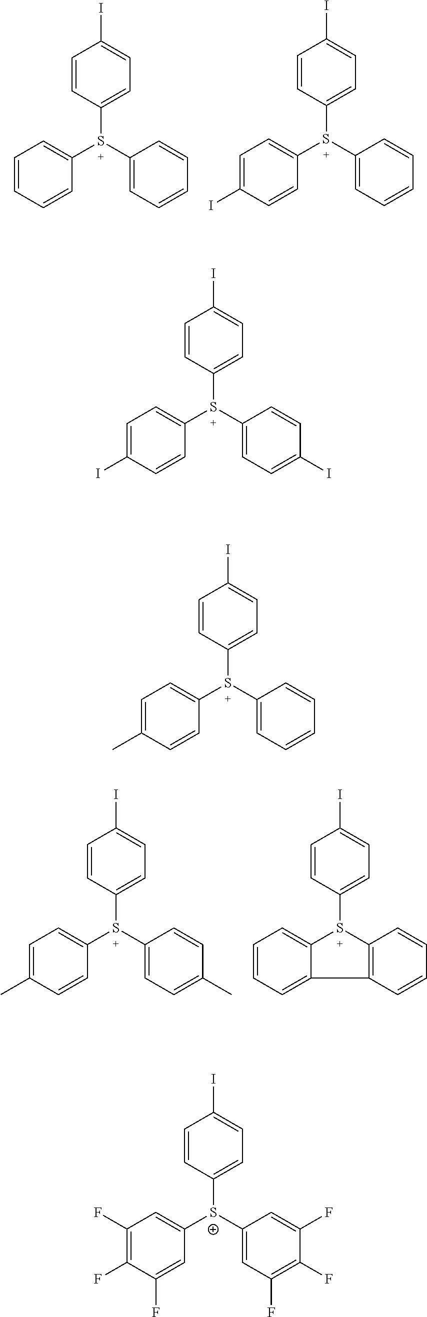

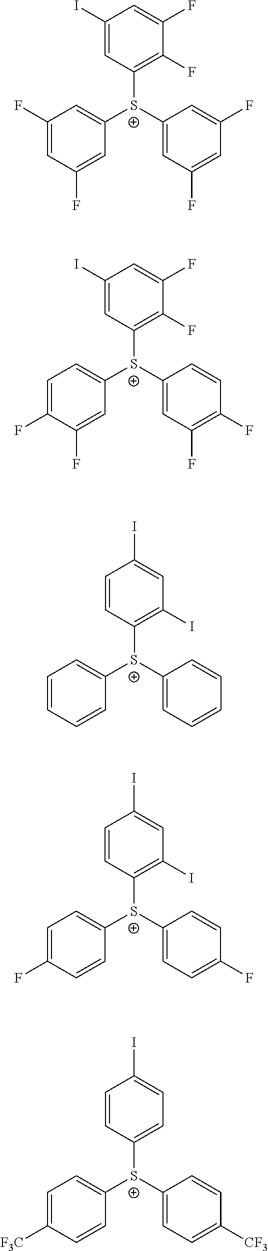 [SMILES: Fc1cc(F)cc([S+](c2cc(F)cc(F)c2)c2cc(I)cc(F)c2F)c1 ; Fc1ccc([S+](c2ccc(F)c(F)c2)c2cc(I)cc(F)c2F)cc1F ; Ic1ccc([S+](c2ccccc2)c2ccccc2)c(I)c1 ; Fc1ccc([S+](c2ccc(F)cc2)c2ccc(I)cc2I)cc1 ; FC(F)(F)c1ccc([S+](c2ccc(I)cc2)c2ccc(C(F)(F)F)cc2)cc1]

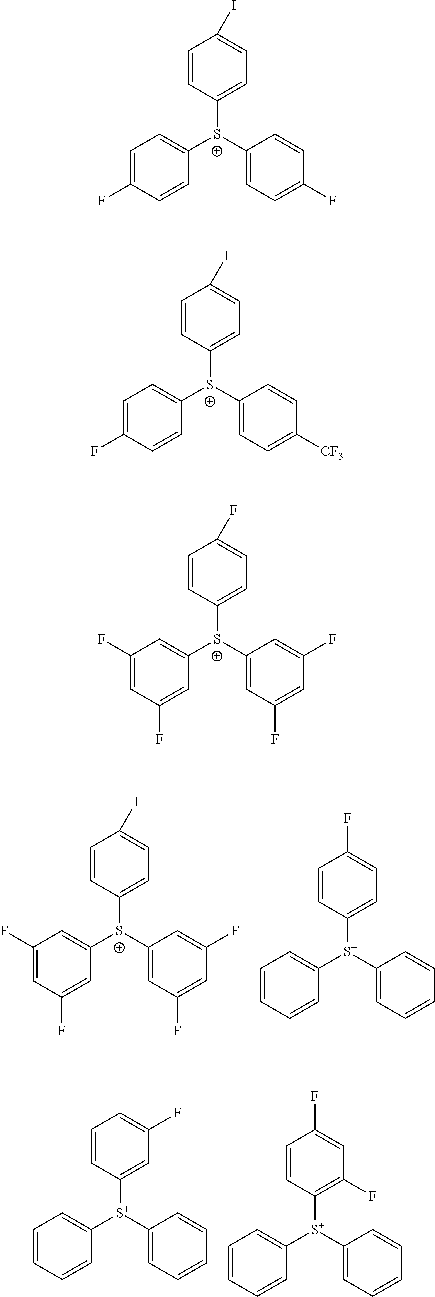

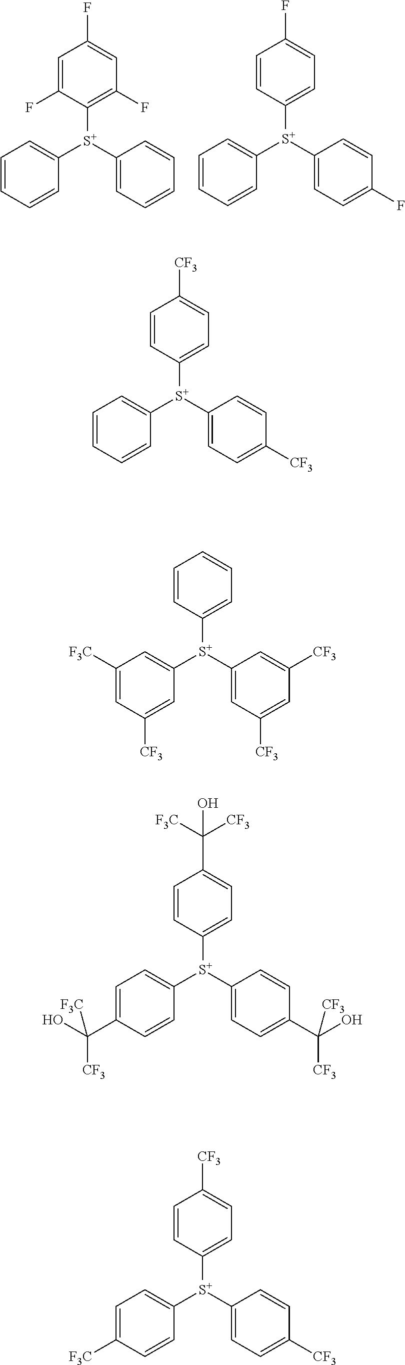 [SMILES: Fc1cc(F)c([S+](c2ccccc2)c2ccccc2)c(F)c1 ; Fc1ccc([S+](c2ccccc2)c2ccc(F)cc2)cc1 ; FC(F)(F)c1ccc([S+](c2ccccc2)c2ccc(C(F)(F)F)cc2)cc1 ; FC(F)(F)c1cc([S+](c2ccccc2)c2cc(C(F)(F)F)cc(C(F)(F)F)c2)cc(C(F)(F)F)c1 ; OC(c1ccc([S+](c2ccc(C(O)(C(F)(F)F)C(F)(F)F)cc2)c2ccc(C(O)(C(F)(F)F)C(F)(F)F)cc2)cc1)(C(F)(F)F)C(F)(F)F ; FC(F)(F)c1ccc([S+](c2ccc(C(F)(F)F)cc2)c2ccc(C(F)(F)F)cc2)cc1]

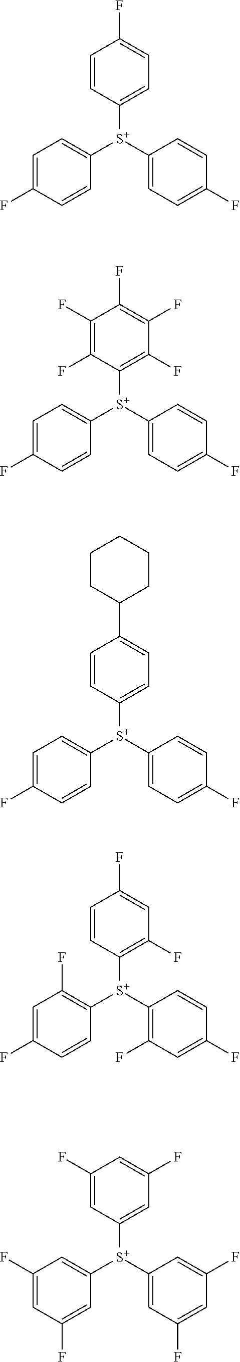 [SMILES: Fc1ccc([S+](c2ccc(F)cc2)c2ccc(F)cc2)cc1 ; Fc1ccc([S+](c2ccc(F)cc2)c2c(F)c(F)c(F)c(F)c2F)cc1 ; Fc1ccc([S+](c2ccc(F)cc2)c2ccc(C3CCCCC3)cc2)cc1 ; Fc1ccc([S+](c2ccc(F)cc2F)c2ccc(F)cc2F)c(F)c1 ; Fc1cc(F)cc([S+](c2cc(F)cc(F)c2)c2cc(F)cc(F)c2)c1]

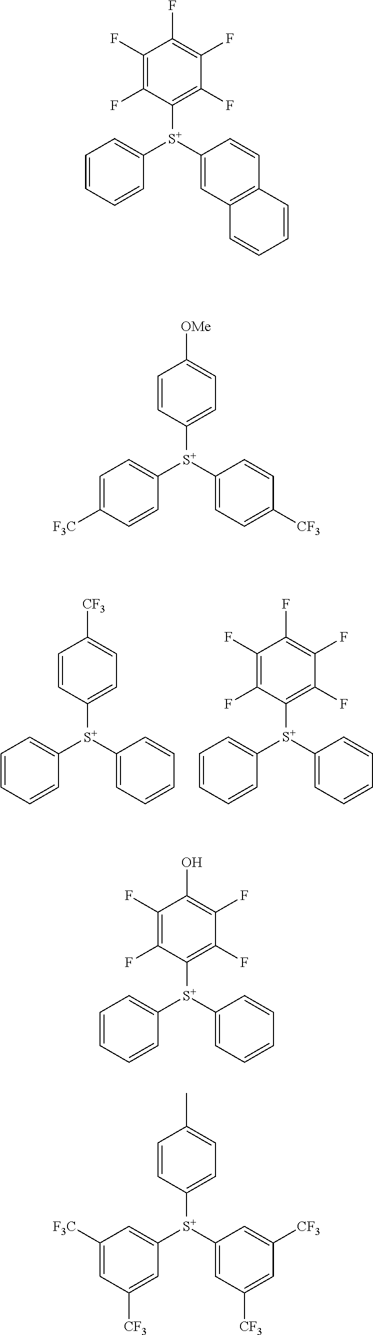 [SMILES: Fc1c(F)c(F)c([S+](c2ccccc2)c2ccc3ccccc3c2)c(F)c1F ; COc1ccc([S+](c2ccc(C(F)(F)F)cc2)c2ccc(C(F)(F)F)cc2)cc1 ; FC(F)(F)c1ccc([S+](c2ccccc2)c2ccccc2)cc1 ; Fc1c(F)c(F)c([S+](c2ccccc2)c2ccccc2)c(F)c1F ; Oc1c(F)c(F)c([S+](c2ccccc2)c2ccccc2)c(F)c1F ; Cc1ccc([S+](c2cc(C(F)(F)F)cc(C(F)(F)F)c2)c2cc(C(F)(F)F)cc(C(F)(F)F)c2)cc1]

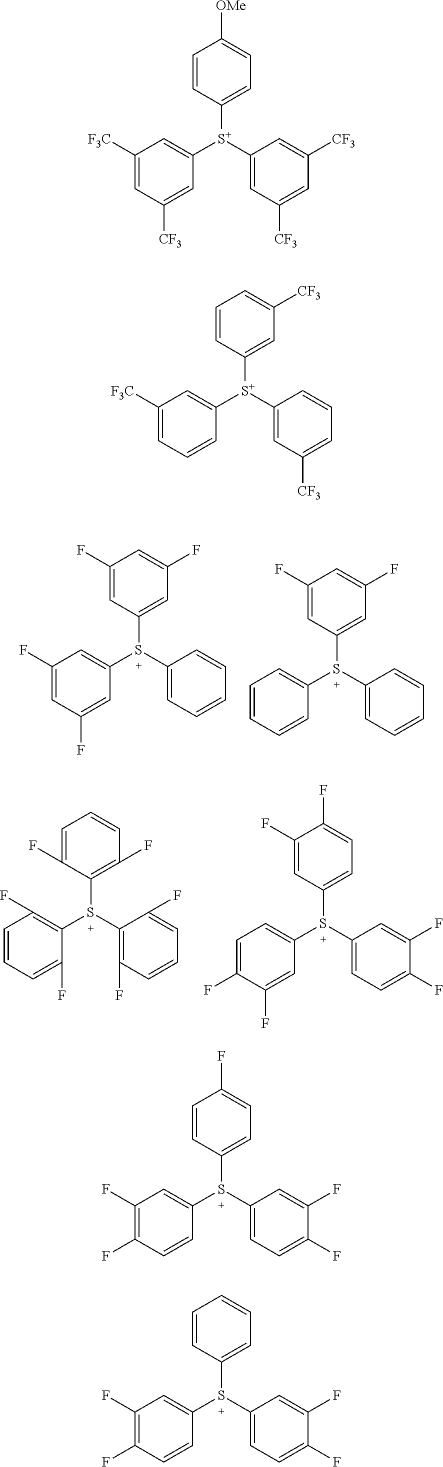

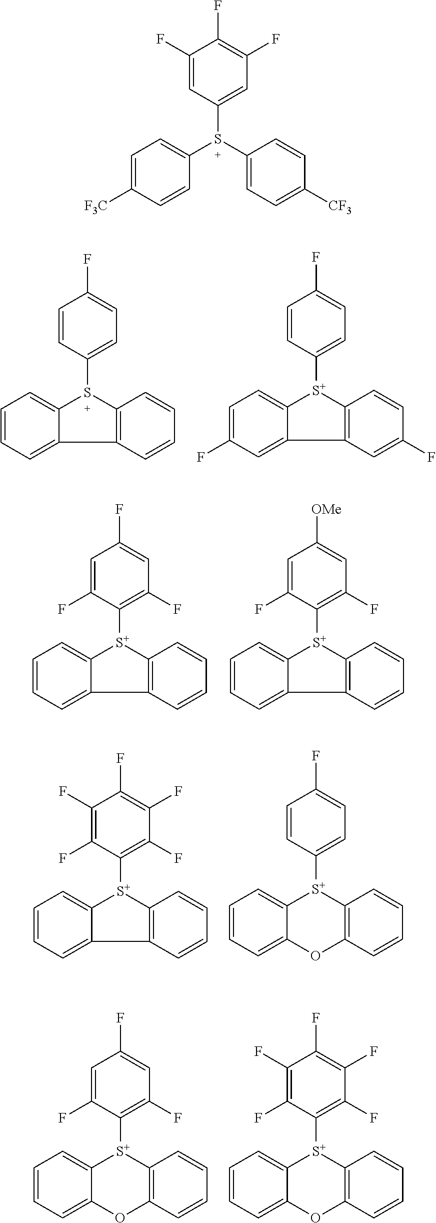

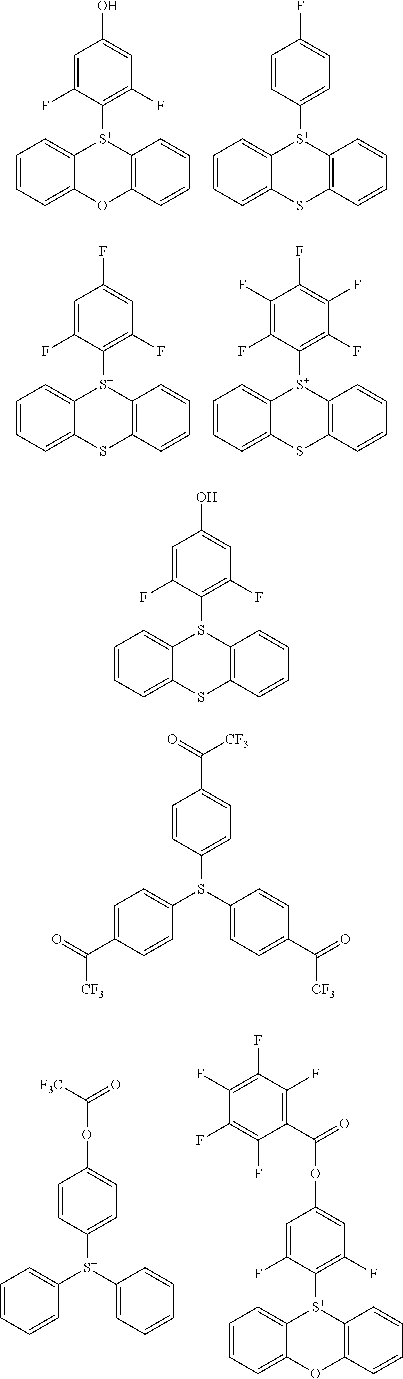

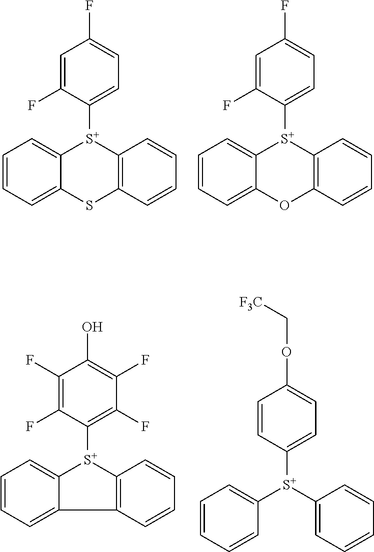 [SMILES: Fc1ccc([S+]2c3ccccc3Sc3ccccc32)c(F)c1 ; Fc1ccc([S+]2c3ccccc3Oc3ccccc32)c(F)c1 ; Oc1c(F)c(F)c(-[s+]2c3ccccc3c3ccccc32)c(F)c1F ; FC(F)(F)COc1ccc([S+](c2ccccc2)c2ccccc2)cc1]

As a side chain structure of the base polymer, an aspect can also be suitably employed in which the first onium cation is bonded to the main chain, and the first organic acid anion is ionically bonded to the first onium cation as a counter ion of the first onium cation. In this case, it is preferable that the first onium cation be bonded to the main chain via a divalent linking group or a single bond, and a structure from V² to SO₃⁻ in the formula (a1) be ionically bonded to the first onium cation as a counter ion. As the divalent linking group, the group represented by L¹¹ of the formula (1) can be suitably employed.

When the base polymer contains the structural unit (II), the lower limit of the content ratio of the structural unit (II) (the total content ratio when a plurality of structural units (II) are contained) is preferably 1 mol %, more preferably 5 mol %, and still more preferably 8 mol % based on all structural units each constituting the base polymer. The upper limit of the content ratio is preferably 30 mol %, more preferably 20 mol %, and still more preferably 15 mol %. When the content ratio of the structural unit (II) is within the above range, the function as an acid generating structure can be sufficiently exhibited, and the various resist properties can be exhibited.

The monomers that afford the structural unit (II-1) can be synthesized, for example, by the same method as that for a sulfonium salt having a polymerizable anion described in JP-B-5201363.

### (Structural Unit (III))

A structural unit (III) is a structural unit that has a phenolic hydroxyl group (excluding structures that fall under structural units (I) and (II)). When the polymer contains the structural unit (IV), the solubility into a developer can be more appropriately adjusted, and as a result, the sensitivity and the like of the radiation-sensitive composition can be further improved. When KrF excimer laser light, EUV, an electron beam, or the like is used as radiation to be applied in an exposure step in a resist pattern forming method, the structural unit (IV) contributes to improvement in etching resistance and improvement in difference in solubility into a developer (dissolution contrast) between an exposed area and an unexposed area. In particular, the polymer containing the structural unit (III) can be suitably applied for pattern formation using exposure with radiation having a wavelength of 50 nm or less, such as an electron beam or EUV. The structural unit (III) is preferably represented by formula (2).

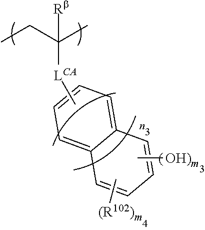

(In the formula (2),
R^{β} is a hydrogen atom, a fluorine atom, a methyl group, or a trifluoromethyl group.
L^{CA} is a single bond, —COO—*, or —O—*. * is a bond on the aromatic ring side.
R¹⁰² is a halogen atom, a cyano group, a nitro group, an alkyl group, an alkoxycarbonyl group, an acyl group, or an acyloxy group. When there are a plurality of R¹⁰²s, the plurality of R¹⁰²s are the same as or different from each other.
n₃ is an integer of 0 to 2, m₃ is an integer of 1 to 8, and my is an integer of 0 to 8, provided that 1≤m₃+m₄≤2n₃+5 is satisfied.)
R^{β} is preferably a hydrogen atom or a methyl group from the viewpoint of the copolymerizability of a monomer that affords the structural unit (III).
L^{CA} is preferably a single bond or —COO—*.

As the halogen atom, the alkyl group, the fluorinated alkyl group, the alkoxycarbonyloxy group, the acyl group, or the acyloxy group as R¹⁰², the groups listed as the substituent of L¹ of the above formula (1) can be suitably employed. As the halogen atom as R¹⁰², an iodine atom is preferable.

The n₃ is more preferably 0 or 1, still more preferably 0.

m₃ is preferably an integer of 1 to 3, more preferably 1 or 2.

m₄ is preferably an integer of 0 to 3, more preferably an integer of 0 to 2.

As the structural unit (III), structural units represented by formulas (2-1) to (2-20) (hereinafter also referred to as “structural units (2-1) to (2-20)”) and the like are preferable.

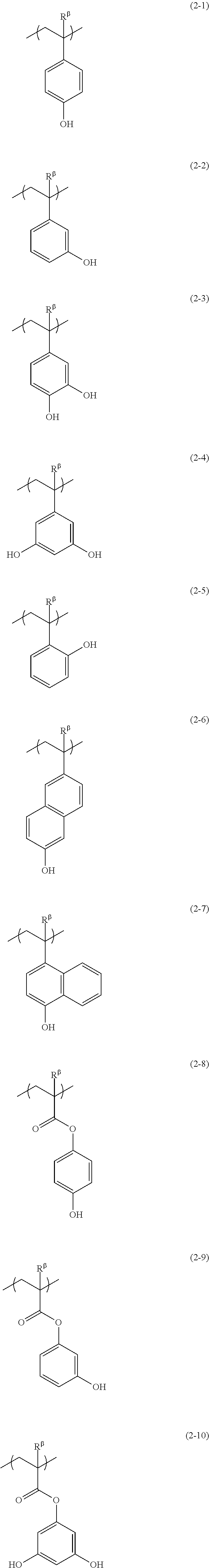

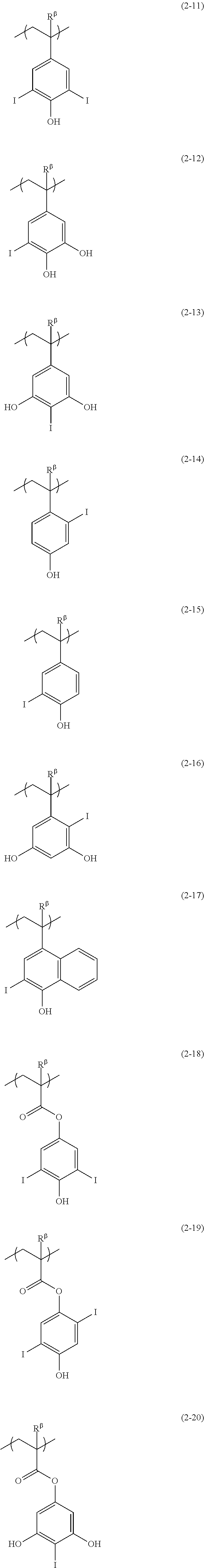

In the formulas (2-1) to (2-20), R^{β} is the same as in the formula (2).

The lower limit of the content ratio of the structural unit (III) (the total content ratio is used when a plurality of structural units (III) are present) is preferably 5 mol %, more preferably 10 mol %, and still more preferably 15 mol % based on all structural units constituting the base polymer. The upper limit of the content ratio is preferably 90 mol %, more preferably 80 mol %, and still more preferably 70 mol %. When the content ratio of the structural unit (III) is adjusted within the above range, the radiation-sensitive composition can further improve the sensitivity and development contrast.

### (Structural Unit (IV))

The base polymer optionally has other structural units. Examples of the other structural units include a structural unit (IV) containing a polar group (excluding those corresponding to the structural units (I) to (III) and a structural unit (V) described later). When the base polymer further has the structural unit (IV), the solubility of the base polymer in a developer can be adjusted, and as a result, the lithographic performance, such as resolution, of the radiation-sensitive composition can be improved. Examples of the polar group include a hydroxy group, a carboxy group, a cyano group, a nitro group, and a sulfonamide group. Among them, a hydroxy group and a carboxy group are preferable, and a hydroxy group is more preferable.

Examples of the structural unit (IV) include structural units represented by formulas.

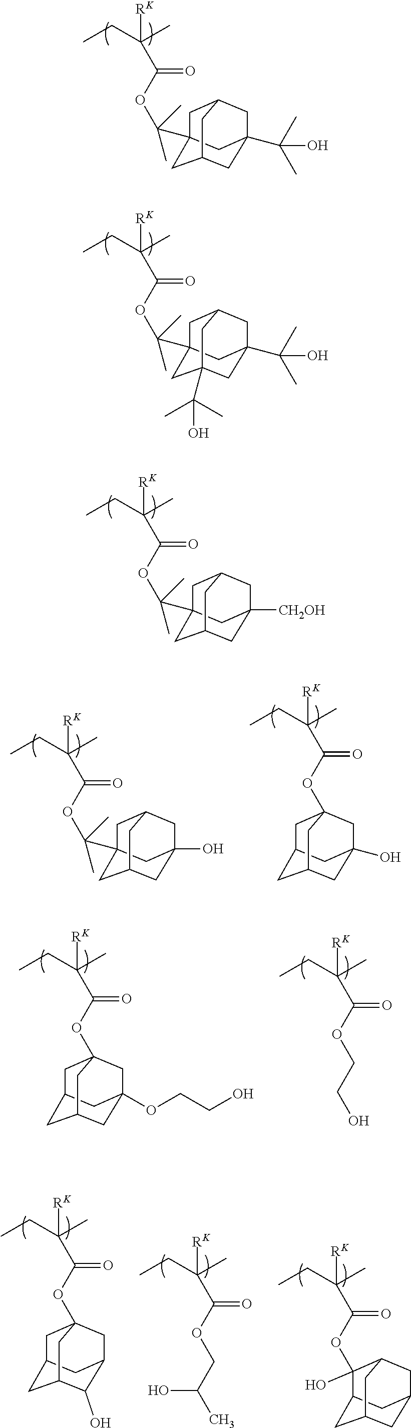

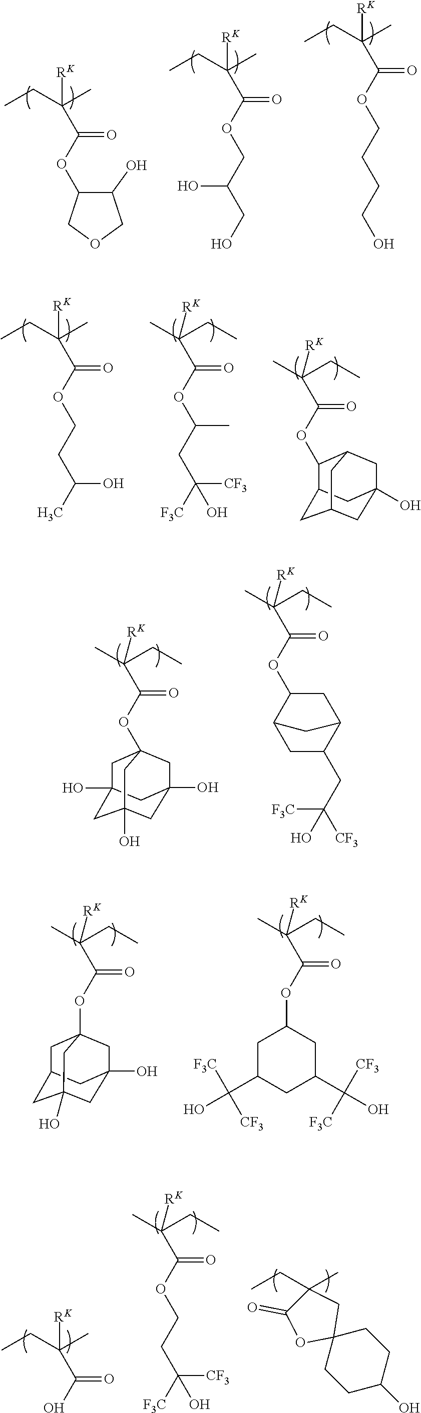

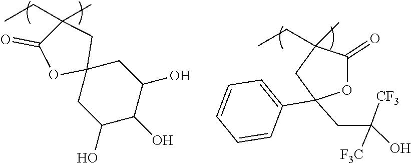

In the formulas, R^{K} is a hydrogen atom, a fluorine atom, a methyl group, or a trifluoromethyl group.

When the base polymer has the structural unit (IV) having a polar group, the lower limit of the content ratio of the structural unit (IV) is preferably 1 mol %, more preferably 5 mol %, and still more preferably 8 mol % based on all structural units constituting the base polymer. The upper limit of the content ratio is preferably 30 mol %, more preferably 20 mol %, and still more preferably 15 mol %. When the content ratio of the structural unit (IV) is adjusted within the above range, the lithographic performance, such as resolution, of the radiation-sensitive composition can further be improved.

### (Structural Unit (V))

The structural unit (V) as another structural unit is a structural unit containing at least one structure selected from the group consisting of a lactone structure, a cyclic carbonate structure, and a sultone structure. When the base polymer further has the structural unit (V), the solubility of the base polymer in a developer can be adjusted, and as a result, the lithographic performance, such as resolution, of the radiation-sensitive composition can be improved. The adhesion between a resist pattern formed from the base polymer and a substrate can also be improved.

The lower limit of the content ratio of the structural unit (V) is preferably 1 mol %, more preferably 2 mol %, and still more preferably 3 mol % based on all structural units constituting the base polymer. The upper limit of the content ratio is preferably 20 mol %, more preferably 16 mol %, and still more preferably 12 mol %. When the content ratio of the structural unit (V) is adjusted within the above range, the lithographic performance, such as resolution, of the radiation-sensitive composition and the adhesion between a resist pattern to be formed and a substrate can be further improved.

The polymer may contain a structural unit derived from styrene or iodostyrene (hereinafter, also referred to as “structural unit (VI)”) as an additional structural unit. When the base polymer has the structural unit (VI) having a polar group, the lower limit of the content ratio of the structural unit (VI) is preferably 1 mol %, more preferably 2 molo, and still more preferably 3 mol % based on all structural units constituting the base polymer. The upper limit of the content ratio is preferably 15 mol %, more preferably 10 mol %, and still more preferably 8 mol %.

The base polymer preferably contains an iodo group. In this case, the iodo group may be contained in any one or more of the structural units (I) to (VI). When the base polymer contains an iodo group, the base polymer can exhibit excellent sensitivity and CDU.

### (Method for Synthesizing Base Polymer)

The base polymer can be synthesized by, for example, polymerizing monomers that will afford respective structural units in an appropriate solvent using a radical polymerization initiator or the like.

The molecular weight of the base polymer is not particularly limited, but the lower limit of the weight average molecular weight (Mw) as determined by Gel Permeation Chromatography (GPC) relative to standard polystyrene is preferably 3,000, more preferably 4,000, still more preferably 5,000. The upper limit of the Mw is preferably 20,000, more preferably 12,000, still more preferably 8,000. When the Mw of the base polymer is adjusted to within the above range, the resulting resist film can exhibit good heat resistance and developability.

The ratio (Mw/Mn) of the Mw to the number average molecular weight (Mn) of the base polymer as determined by GPC relative to standard polystyrene is usually 1 or more and 5 or less, preferably 1 or more and 3 or less, more preferably 1 or more and 2 or less.

The method for measuring Mw and Mn of a polymer in the specification is as described in Examples.

The lower limit of the content ratio of the base polymer is preferably 50% by mass, more preferably 60% by mass, and still more preferably 70% by mass based on the total solid content of the radiation-sensitive composition. The upper limit of the content ratio is preferably 98% by mass, more preferably 95% by mass, and still more preferably 92% by mass.

### <Other Polymers>

The radiation-sensitive composition according to the present embodiment may contain, as another polymer, a polymer having higher content by mass of fluorine atoms than the above-described base polymer (hereinafter, also referred to as “high fluorine-content polymer”). When the radiation-sensitive composition includes the high fluorine-content polymer, the high fluorine-content polymer can be localized in the surface layer of a resist film compared to the base polymer, and as a result, the state of the surface of the resist film and the component distribution in the resist film can be controlled to a desired state.

### (Method for Synthesizing High Fluorine-Content Polymer)

The high fluorine-content polymer can be synthesized by the same method as the method for synthesizing the base polymer described above.

### <Radiation-Sensitive Acid Generator>

The radiation-sensitive acid generator contains a second organic acid anion and a second onium cation liberated from the second organic acid anion, and forms the onium salt structure. The radiation-sensitive acid generator is a component that generates an acid through exposure to light. The acid generated through exposure to light has a function of dissociating the acid-dissociable group of the base polymer to generate a carboxy group or the like. The radiation-sensitive acid generator has the form in which the onium salt structure exists alone as a low molecular compound (liberated from the polymer), and is different from the radiation-sensitive acid generating structure in which the first organic acid anion or the first onium cation is bonded (covalently bonded) to a main chain as a side chain structure of the base polymer as in the structural unit (II) in the base polymer.

The radiation-sensitive acid generator includes a compound represented by formula (1) (hereinafter, also referred to as “compound (1)”):

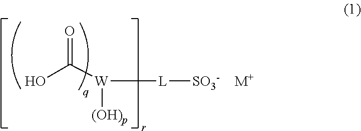

wherein in the formula (1),
W is a (p+q+1)-valent aromatic ring having 5 to 20 ring members, when there are a plurality of Ws, the plurality of Ws are identical to or different from each other,
L is a (r+1)-valent linking group,
r is an integer of 1 to 3,
p is an integer of 0 to 3, when there are a plurality of ps, the plurality of ps are identical to or different from each other,
q is an integer of 1 to 3, when there are a plurality of qs, the plurality of qs are identical to or different from each other, and
M⁺ is a monovalent onium cation.

As the aromatic ring having 5 to 20 ring members in W, rings corresponding to 5 to 20 ring members among the aromatic rings in the iodo group-containing aromatic ring structure can be suitably employed. Examples of the (p+q+1)-valent aromatic ring having 5 to 20 ring members represented by W include a structure obtained by removing (p+q) hydrogen atoms from the aromatic ring. Among them, W is preferably a benzene ring.

As the substituent in the case where W has a substituent, a substituent (provided that a carboxy group and a hydroxy group are excluded) that can be possessed by the substituent that can be possessed by the alkanediyl group or the arenediyl group represented by L^{11a} in the formula (1) can be suitably employed. As the substituent in W, a halogen atom is preferable, and a fluorine atom or an iodine atom is more preferable.

In the formula (1), p and r preferably satisfy the following relationship.

When r is 1, p is an integer of 1 to 3; or
when r is greater than or equal to 2, at least one p is an integer of 1 to 3.

When p and r satisfy the relationship, it is preferable that at least one —OH be bonded to a carbon atom adjacent to a carbon atom to which —COOH of the formula (1) is bonded (that is, —OH be present at the ortho position with respect to —COOH on the aromatic ring).

When p and r satisfy the relationship, a structure in which —COOH and —OH in the formula (1) are bonded to the same aromatic ring is obtained. This makes it possible to improve the effect of controlling the basicity of the acid diffusion controlling agent by acid release, thereby enhancing the storage stability of the composition. Furthermore, satisfying the positional relationship between —OH and —COOH (ortho position relationship) can further improve the storage stability.
r is preferably 1 or 2, more preferably 1.

As the (r+1)-valent linking group in L, a group obtained by removing r hydrogen atoms from the monovalent organic group having 1 to 20 carbon atoms represented by X¹ and X² in the formula (P1) can be suitably employed.

L preferably includes at least one structure selected from the group consisting of a cyclic structure, an ester bond, and an ether bond. As the cyclic structure in L, the cyclic structure shown in the first organic acid anion can be suitably employed.

In the formula (1), a fluorine atom or a fluorinated hydrocarbon group is preferably bonded to a carbon atom adjacent to a sulfur atom of —SO₃—. As a result, the acid generated through exposure to light can have sufficient acidity to exhibit good sensitivity and CDU.

Examples of the organic acid anion (corresponding to the second organic acid anion) of the compound (1) include, but are not limited to, the structures shown below.

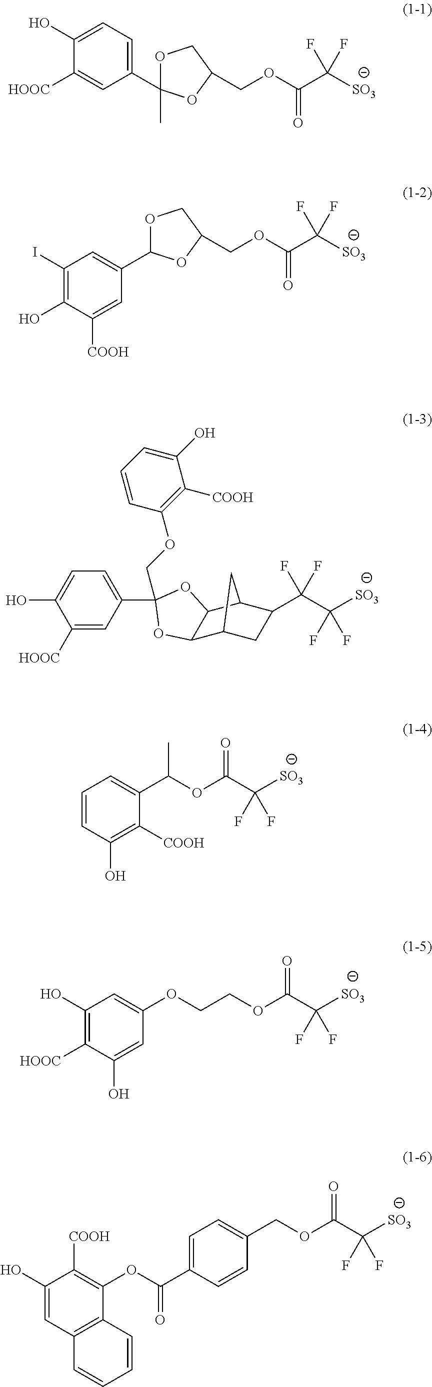

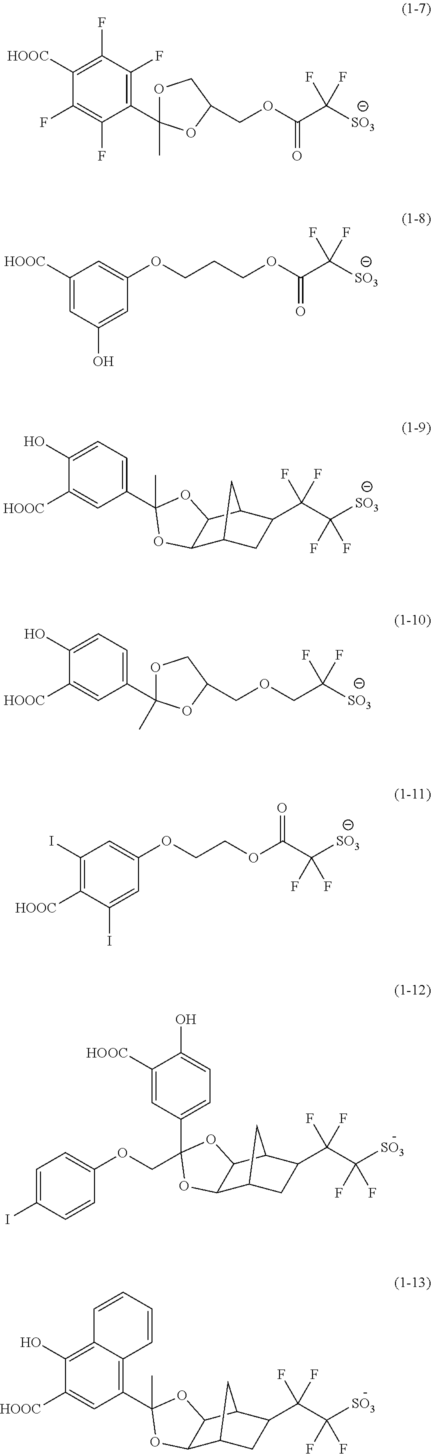

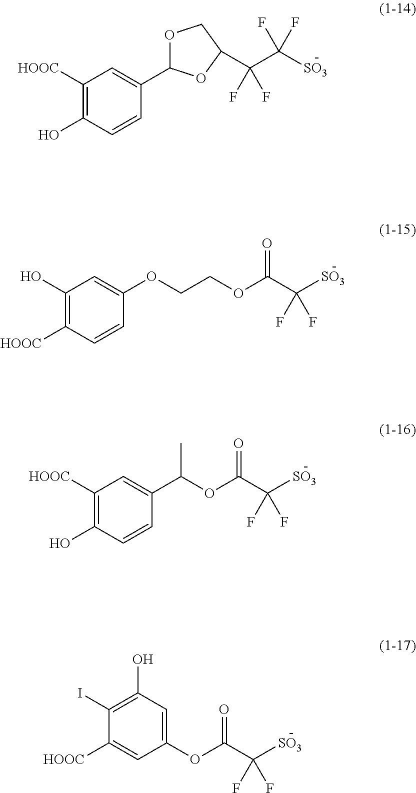

As the structure of the monovalent onium cation (corresponding to the second onium cation) of the compound (1), the structure of the first onium cation of the structural unit (II) in the base polymer can be suitably employed.

The radiation-sensitive acid generator may contain a radiation-sensitive acid generator different from the compound (1) (hereinafter, also referred to as “another radiation-sensitive acid generator”). Suitable examples of another radiation-sensitive acid generator include a compound having a structure obtained by removing-COOH from the formula (1) and a known radiation-sensitive acid generator.

In the case of the contained aspects (2) and (5) to (7), at least a part of the second onium cation (including the onium cation of the compound (1)) is an iodonium cation. That is, the contained aspect of the iodonium cation in the radiation-sensitive acid generator when the composition does not contain another radiation-sensitive acid generator is an aspect in which the compound (1) has an iodonium cation. When the composition contains another radiation-sensitive acid generator, the contained aspect of the iodonium cation is any of an aspect in which the compound (1) has an iodonium cation and the other radiation-sensitive acid generator has no iodonium cation, an aspect in which the compound (1) has no iodonium cation and the other radiation-sensitive acid generator has an iodonium cation, and an aspect in which the compound (1) has an iodonium cation and the other radiation-sensitive acid generator has an iodonium cation. In the case of contained aspects other than these contained aspects, the second onium cation is another cation.

Among them, an aspect in which the composition contains another radiation-sensitive acid generator, the compound (1) has no an iodonium cation, and the other radiation-sensitive acid generator has an iodonium cation is preferable. As a result, the storage stability can be further improved.

The radiation-sensitive acid generator can also be synthesized by a publicly known method, particularly by a salt exchange reaction. A publicly known radiation-sensitive acid generator may also be used as long as the effects of the present disclosure are not impaired.

These radiation-sensitive acid generators may be used singly, or two or more thereof may be used in combination. When the radiation-sensitive composition contains the radiation-sensitive acid generator, the lower limit of the content of the radiation-sensitive acid generator (the total when two or more is contained) is preferably 5 parts by mass, more preferably 8 parts by mass, and still more preferably 10 part by mass based on 100 parts by mass of the base polymer. The upper limit of the content is preferably 60 parts by mass, more preferably 50 parts by mass, and still more preferably 45 parts by mass. This makes it possible to exhibit excellent sensitivity in resist pattern formation.

### <Acid Diffusion Controlling Agent>

The acid diffusion controlling agent contains a third organic acid anion and a third onium cation liberated from the third organic acid anion and generates an acid which does not dissociate the acid-dissociable group through exposure to light. The acid diffusion controlling agent has a function of suppressing the diffusion of the acid generated from the acid generation structure in the case of containing the structural unit (II) or the radiation-sensitive acid generator in the unexposed portion by salt exchange. As a result, the diffusion of the acid in the unexposed portion can be suppressed, thereby forming a resist pattern excellent in resolution and development contrast.

At least one selected from the group consisting of the third organic acid anion and the third onium cation preferably has an iodo group, more preferably has the iodo group-containing aromatic ring structure.

The structure of the third organic acid anion is not specified, but preferably includes —O—, —CO—, a cyclic structure, or a combination thereof. As the cyclic structure, the cyclic structure in the first organic acid anion of the structural unit (II) of the base polymer can be suitably employed.

In the acid diffusion controlling agent, the third organic anion preferably has a sulfonate anion or a carboxylate anion as the acid anion moiety (provided that when the third organic acid anion has the sulfonate anion, neither a fluorine atom nor a fluorinated hydrocarbon group is bonded to a carbon atom adjacent to a sulfur atom of the sulfonate anion). As a result, the acid diffusion controlling agent can efficiently exhibit the above function.

Examples of the acid diffusion controlling agent include a sulfonium salt compound represented by formula (8-1) and an iodonium salt compound represented by formula (8-2).

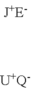

In the formulas (8-1) to (8-2), J⁺ is a sulfonium cation, and U⁺ is an iodonium cation. E⁻ and Q⁻ are each independently the third organic acid anion represented by OH⁻, R^{α}—COO—, or R^{α}—SO₃⁻. R^{α} is a monovalent organic group having 1 to 30 carbon atoms. As the organic group, the monovalent organic groups represented by X¹ and X² in the formula (P1) having, instead of 1 to 20 carbon atoms, an expanded range of carbon atoms of 1 to 30 can suitably be employed.

Examples of the fourth organic acid anion of the acid diffusion controlling agent include, but are not limited to, those shown below. As the organic acid anion having no iodo group-containing aromatic ring structure, a structure in which an iodo group in formula is substituted with an atom or a group other than the iodo group such as a hydrogen atom or another substituent can be suitably employed.

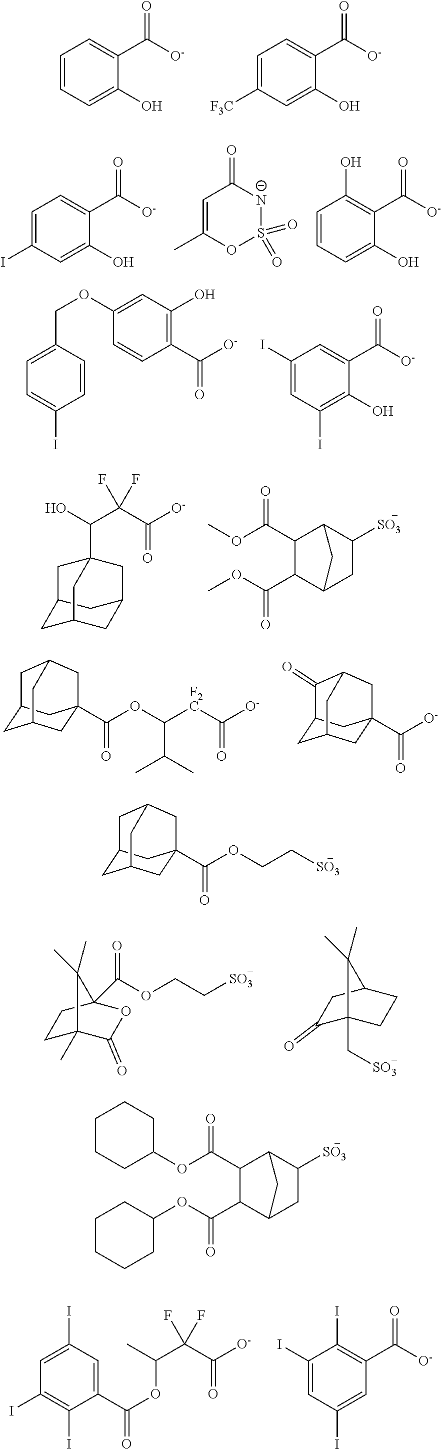

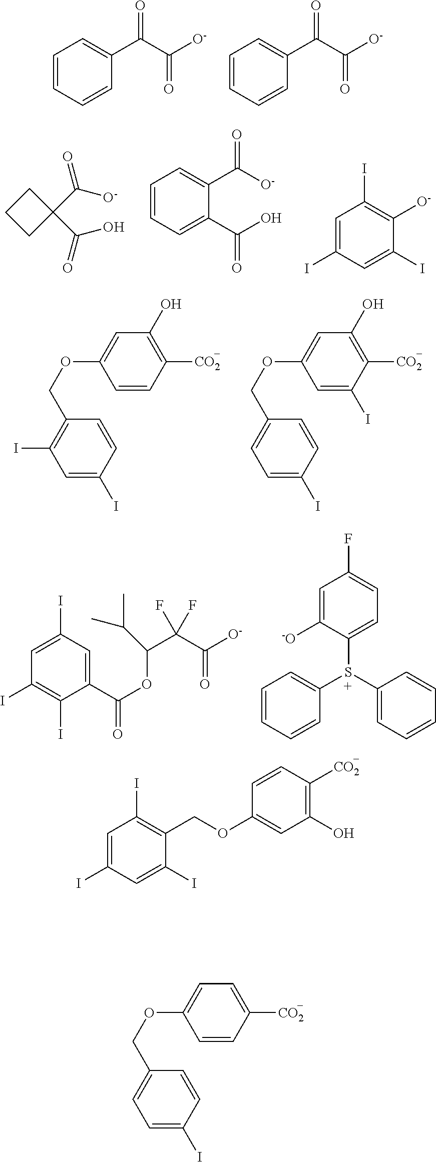

As the third onium cation in the acid diffusion controlling agent, the structure of the first onium cation of the structural unit (II) in the base polymer can be suitably employed. In the case of the contained aspects (3), (4), (6) and (7), the third onium cation is an iodonium cation. In the case of contained aspects other than these contained aspects, the third onium cation is another cation.

The acid diffusion controller can also be synthesized by known methods, particularly by salt exchange reaction.

These acid diffusion controlling agents may be used singly, or two or more thereof may be used in combination.

The lower limit of the content of the acid diffusion controlling agent (the total content in the case of a plurality of acid diffusion controlling agents) is preferably 5 mol %, more preferably 10 mol %, still more preferably 15 mol % based on the total of the content of a monomer corresponding to the structural unit (II) of the base polymer and the content of the radiation-sensitive acid generator. The upper limit of the content is preferably 40 mol %, more preferably 30 mol %, still more preferably 25 mol %.

### <Solvent>

The radiation-sensitive composition according to the present embodiment contains a solvent. The solvent is not particularly limited as long as the solvent is one capable of dissolving or dispersing the base polymer, the radiation-sensitive acid generator, the acid diffusion controlling agent, and additives or the like contained as desired.

Examples of other solvents include an alcohol-based solvent, an ether-based solvent, a ketone-based solvent, an amide-based solvent, an ester-based solvent, and a hydrocarbon-based solvent.

Examples of the alcohol-based solvent include:
monoalcohol-based solvents having 1 to 18 carbon atoms, such as iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
polyhydric alcohol-based solvents having 2 to 18 carbon atoms, such as ethylene glycol, 1,2-propylene glycol, 2-methyl-2,4-pentanediol, 2,5-hexanediol, diethylene glycol, dipropylene glycol, triethylene glycol, and tripropylene glycol; and
partially etherized polyhydric alcohol-based solvents obtained by etherifying some of hydroxy groups of the polyhydric alcohol-based solvent.

In the present embodiment, the alcohol-based solvents also include alcohol acid ester-based solvents such as methyl lactate, ethyl lactate, propyl lactate, butyl lactate, methyl 2-hydroxyisobutyrate, i-propyl 2-hydroxyisobutyrate, i-butyl 2-hydroxyisobutyrate, and n-butyl 2-hydroxyisobutyrate.

Examples of the ether-based solvent include:
dialkyl ether-based solvents, such as diethyl ether, dipropyl ether, and dibutyl ether;
cyclic ether-based solvents, such as tetrahydrofuran and tetrahydropyran;
aromatic ring-containing ether-based solvents, such as diphenyl ether and anisole (methyl phenyl ether); and
etherized polyhydric alcohol-based solvents obtained by etherifying a hydroxy group of the polyhydric alcohol-based solvent.

Examples of the ketone-based solvent include chain ketone-based solvents, such as acetone, butanone, and methyl-iso-butyl ketone;
cyclic ketone-based solvents, such as cyclopentanone, cyclohexanone, and methylcyclohexanone; and
2,4-pentanedione, acetonylacetone, and acetophenone.

Examples of the amide-based solvent include cyclic amide-based solvents, such as N,N′-dimethylimidazolidinone and N-methylpyrrolidone; and
chain amide-based solvents, such as N-methylformamide, N, N-dimethylformamide, N, N-diethylformamide, acetamide, N-methylacetamide, N, N-dimethylacetamide, and N-methylpropionamide.

Examples of the ester-based solvent include:
monocarboxylic acid ester-based solvents, such as n-butyl acetate;
partially etherized polyhydric alcohol acetate-based solvents, such as diethylene glycol mono-n-butyl ether acetate, propylene glycol monomethyl ether acetate, and dipropylene glycol monomethyl ether acetate;
lactone-based solvents, such as γ-butyrolactone and valerolactone;
carbonate-based solvents, such as diethyl carbonate, ethylene carbonate, and propylene carbonate; and
polyvalent carboxylic acid diester-based solvents, such as propylene glycol diacetate, methoxytriglycol acetate, diethyl oxalate, ethyl acetoacetate, and diethyl phthalate.

Examples of the hydrocarbon-based solvent include:
aliphatic hydrocarbon-based solvents, such as n-hexane, cyclohexane, and methylcyclohexane; and
aromatic hydrocarbon-based solvents, such as benzene, toluene, di-iso-propylbenzene, and n-amylnaphthalene.

Among them, an ester-based solvent, an ether-based solvent, and an alcohol-based solvent are preferable, a partially etherized polyhydric alcohol acetate-based solvent, a partially etherized polyhydric alcohol-based solvent, an alcoholic acid ester-based solvent, and a monoalcohol-based solvent having 1 to 18 carbon atoms are more preferable, and propylene glycol monomethyl ether acetate, propylene glycol monomethyl ether, methyl 2-hydroxyisobutyrate, and diacetone alcohol are still more preferable. The radiation-sensitive composition may contain one solvent, or two or more solvents.

### <Other Optional Components>

The radiation-sensitive composition may contain, in addition to the components, other optional components. Examples of other optional components may include a cross-linking agent, a localization enhancing agent, a surfactant, an alicyclic backbone-containing compound, and a sensitizer. These other optional components may be used singly, or two or more thereof may be used in combination.

### <Method for Preparing Radiation-Sensitive Composition>

The radiation-sensitive composition can be prepared, for example, by mixing the base polymer, the acid diffusion controlling agent and the solvent, and if necessary, the optional component at a prescribed ratio. The radiation-sensitive composition is, after the mixing, preferably filtered through, for example, a filter having a pore size of approximately 0.05 μm to 0.4 μm. The solid matter concentration of the radiation-sensitive composition is usually 0.1% by mass to 50% by mass, preferably 0.5% by mass to 30% by mass, more preferably 18 by mass to 20% by mass.

### <Pattern Forming Method>

A pattern forming method according to the present disclosure includes:
(1) applying the radiation-sensitive composition directly or indirectly on a substrate to form a resist film (hereinafter also referred to as “resist film forming step”);
(2) exposing the resist film to light (hereinafter also referred to as “exposure step”); and
(3) developing the exposed resist film with a developer (hereinafter also referred to as “development step”).

According to the pattern forming method described above, a radiation-sensitive composition is used that exhibits good storage stability and excellent sensitivity and CDU during pattern formation, thus enabling the efficient formation of high-quality resist patterns. The following describes each step.

### [Resist Film Forming Step]

In this step (the step (1)), a resist film is formed from the radiation-sensitive composition. Examples of the substrate on which the resist film is formed may include those traditionally known in the art, including a silicon wafer, silicon dioxide, and a wafer coated with aluminum. An organic or inorganic antireflective film disclosed in, for example, JP-B-6-12452 or JP-A-59-93448 may be formed on the substrate. Examples of an applicating method may include a rotary coating (spin coating), flow casting, and roll coating. After the application, soft baking (SB) may be performed to volatilize the solvent in the coating film, as necessary. The temperature of SB is usually from 60 °C to 160 °C, preferably from 80 °C to 140 °C. The duration of SB is usually from 5 seconds to 600 seconds, preferably from 10 seconds to 300 seconds. The thickness of the resist film formed is preferably from 10 nm to 1,000 nm, more preferably from 10 nm to 500 nm.

Furthermore, when the subsequent exposure step is carried out using radiation having a wavelength of 50 nm or less, it is preferable to use a polymer having the structural unit (III) as the base polymer in the composition.

### [Exposure Step]

In this step (the step (2)), the resist film formed in the resist film forming step as the step (1) is exposed by irradiating with radiation through a photomask. Examples of the radiation to be used for the exposure may include an electromagnetic wave including visible ray, ultraviolet ray, far ultraviolet ray, extreme ultraviolet ray (EUV), X ray, and γ ray; and charged particle radiation such as an electron beam and a ray. Among them, far ultraviolet ray, an electron beam, or EUV is preferable. ArF excimer laser light (wavelength is 193 nm), KrF excimer laser light (wavelength is 248 nm), an electron beam, or EUV is more preferable. An electron beam having a wavelength of 50 nm or less, which is identified as the next generation exposing technology, or EUV is further preferable.

After the exposure, post exposure bake (PEB) is preferably performed to promote the dissociation of the acid-dissociable group of the polymer by an acid generated from the radiation-sensitive acid generator through exposure to light in the exposed part of the resist film. As a result of the PEB, there is generated a difference in solubility into a developer between the exposed area and the unexposed area. The temperature of PEB is usually from 50 °C to 180 °C, preferably from 80 °C to 150 °C. The duration of PEB is usually from 5 seconds to 600 seconds, preferably from 10 seconds to 300 seconds.

### [Development Step]

In this step (the step (3)), the resist film exposed in the exposure step as the step (2) is developed with a developer. By this step, the predetermined resist pattern can be formed. After the development, the resist pattern is generally washed with a rinse solution such as water or alcohol, and then dried.

Examples of the developer used for the development may include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferable, and a 2.38% by mass aqueous TMAH solution is more preferable.

In the case of organic solvent development, examples of the solvent may include organic solvents such as hydrocarbon-based solvents, ether-based solvents, ester-based solvents, ketone-based solvents, and alcohol-based solvents, and solvents containing an organic solvent. Examples of the organic solvent may include one, two or more solvents listed as the solvent for the radiation-sensitive composition. Among them, ester-based solvents and ketone-based solvents are preferable. As the ester-based solvents, acetate-based solvents are preferable, and n-butyl acetate and amyl acetate are more preferable. As the ketone-based solvents, chain ketones are preferable, and 2-heptanone is more preferable. The content of the organic solvent in a developer is preferably 80% by mass or more, more preferably 90% by mass or more, still more preferably 95% by mass or more, particularly preferably 99% by mass or more. Examples of the component other than the organic solvent in the developer may include water and silicone oil.

Examples of the developing method may include a method including dipping a substrate in a tank filled with a developer for a given time (dipping method); a developing method including raising a developer on the surface of a substrate due to surface tension and leaving the raised developer for a given time (paddling method); a method including spraying a developer on the surface of a substrate (spraying method); and a method including injecting a developer on a substrate rolling at a constant rate while scanning a developer injection nozzle at a constant rate (dynamic dispensing method).

### EXAMPLES

Hereinafter, a specific description is made of the present invention with reference to Examples, but the present invention is not limited to the Examples. Physical property values in the Examples were measured as follows.

### [Weight-Average Molecular Weight (Mw) and Number-Average Molecular Weight (Mn)]

Measurement was performed by gel permeation chromatography (GPC) with monodisperse polystyrene standards using GPC columns (G2000HXL×2, G3000HXL×1, and G4000HXL×1) manufactured by Tosoh Corporation, under analysis conditions including a flow rate: 1.0 mL/min, an elution solvent: tetrahydrofuran, a sample concentration: 1.0% by mass, an amount of sample injected: 100 μL, a column temperature: 40 °C, and a detector: a differential refractometer. The degree of dispersion (Mw/Mn) was calculated from the measurement results of Mw and Mn.

### <Synthesis of Polymer [A]>

The monomers used for the synthesis of the respective polymers in the respective Examples and Comparative Examples are shown below.

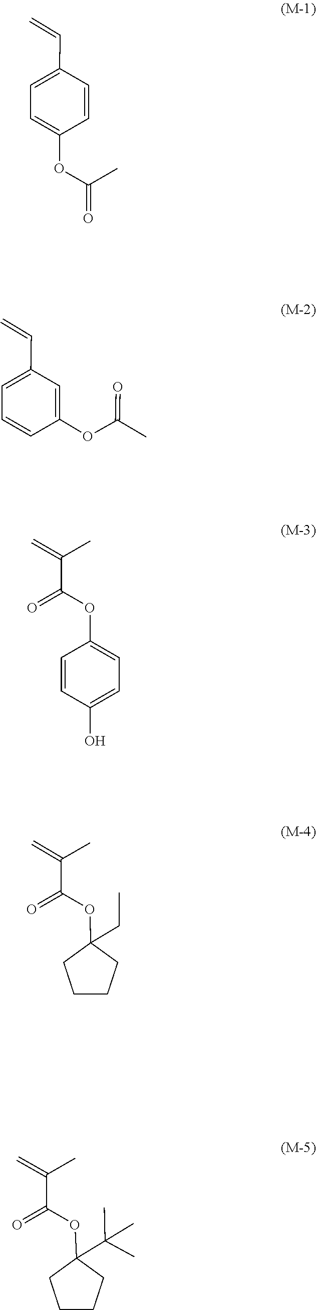

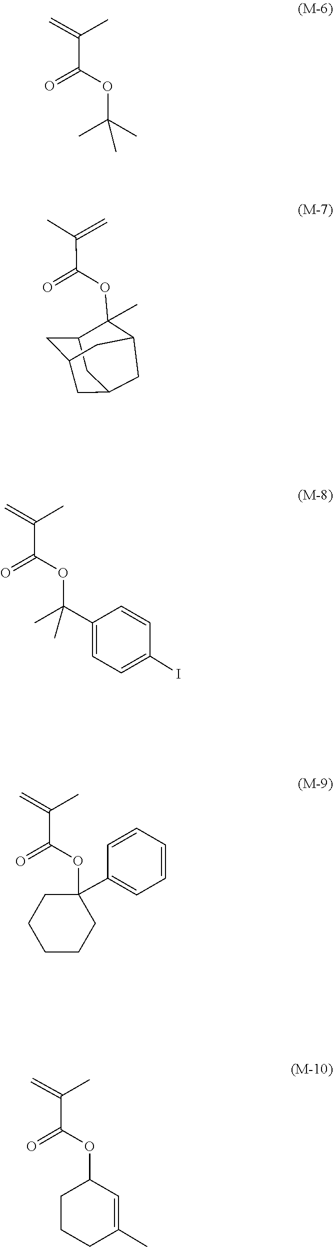

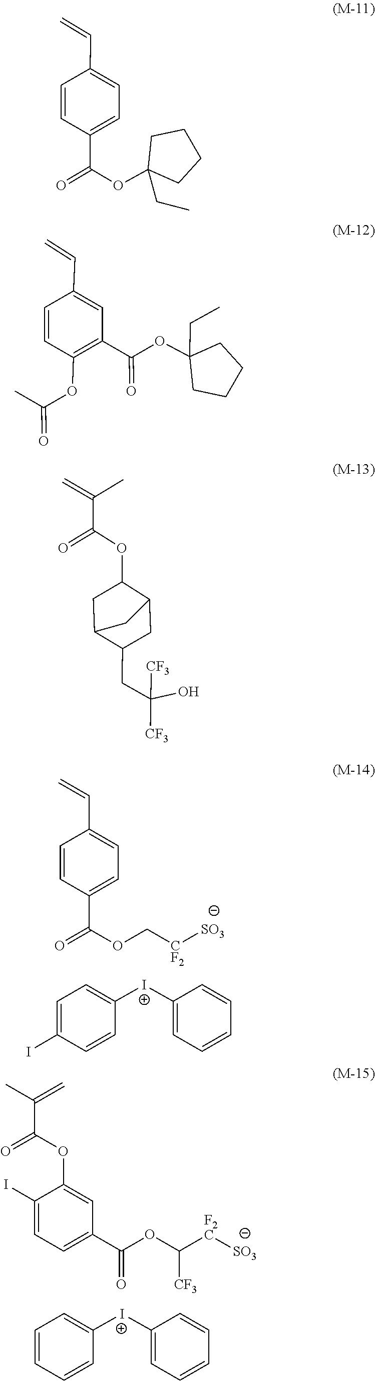

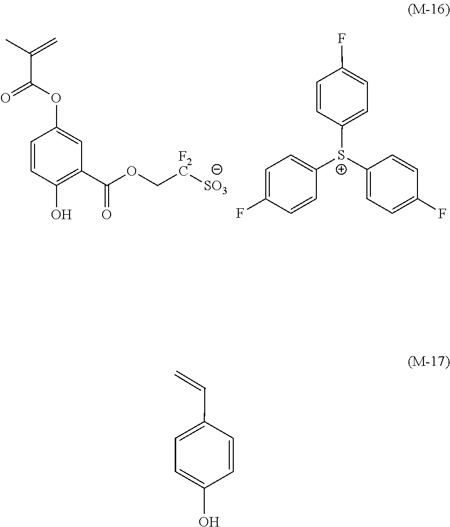

### [Synthesis Example 1] Synthesis of Polymer (A-1)

Compound (M-1) and compound (M-4) were dissolved in 1-methoxy-2-propanol (200 parts by mass based on the total amount of monomers) at a molar ratio of 40/60. Next, azobisisobutyronitrile was added as an initiator in an amount of 6 molo based on all the monomers to prepare a monomer solution. Meanwhile, 1-methoxy-2-propanol (100 parts by mass based on the total amount of monomers) was added to an empty reaction vessel, and was heated to 85 °C with stirring. Next, the monomer solution prepared above was added dropwise over 3 hours, followed by further heating at 85 °C for 3 hours. After completion of the polymerization reaction, the polymerization solution was cooled to room temperature.

The cooled polymerization solution was charged into hexane (500 parts by mass with respect to the polymerization solution), and a precipitated white powder was collected by filtration. The white powder separated by filtration was washed twice with 100 parts by mass of hexane with respect to the polymerization solution, then dissolved in 1-methoxy-2-propanol (300 parts by mass). Next, methanol (500 parts by mass), triethylamine (50 parts by mass), and ultrapure water (10 parts by mass) were added, and a hydrolysis reaction was performed at 70 °C for 6 hours with stirring.

After completion of the reaction, the remaining solvent was distilled off, and the resulting solid was dissolved in acetone (100 parts by mass). This solution was added dropwise into 500 parts by mass of water to permit coagulation of the polymer+. The solid obtained was collected by filtration. The resulting solid was dried at 50 °C for 12 hours, affording a white powdery polymer (A-1).

### Synthesis Examples 2 to 11

Polymers (A-2) to (A-11) were also synthesized in the same manner as in Synthesis Example 1 except that the monomer species and the ratio were changed as shown in Table 1 below.

### Synthesis Example 12

Compound (M-17), compound (M-4), and compound (M-3) were dissolved in 1-methoxy-2-propanol (200 parts by mass based on the total monomer amount) at a molar ratio of 30/60/10. Next, azobisisobutyronitrile was added as an initiator in an amount of 6 mol % based on all the monomers to prepare a monomer solution. Meanwhile, 1-methoxy-2-propanol (100 parts by mass based on the total amount of monomers) was added to an empty reaction vessel, and was heated to 85 °C with stirring. Next, the monomer solution prepared above was added dropwise over 3 hours, followed by further heating at 85 °C for 3 hours. After completion of the polymerization reaction, the polymerization solution was cooled to room temperature.

The cooled polymerization solution was charged into hexane (500 parts by mass with respect to the polymerization solution), and a precipitated white powder was collected by filtration. The white powder collected by filtration was washed twice with 100 parts by mass of hexane relative to the polymerization solution, and then dried at 50 °C for 12 hours, affording a white powdery polymer (A-12).

### Synthesis Examples 13 to 15

Polymers (A-13) to (A-15) were also synthesized in the same manner as in Synthesis Example 12 except that the monomer species and the ratio were changed as shown in Table 1 below. In Table 1 below, “-” indicates that the corresponding ingredient was not used. The same applies to subsequent tables.

TABLE 1

| | | | | | | Monomer that | | Monomer that | | | |
|---|---|---|---|---|---|---|---|---|---|---|---|
| | | Monomer that | | Monomer that | | affords | | affords | | | |
| | | affords structural | | affords structural | | structural | | structural | | | |
| | | unit (III) | | unit (I) | | unit (II) | | unit (IV) | | | |
| | | | Amount | | Amount | | Amount | | Amount | | |
| | | | used | | used | | used | | used | | |
| | Polymer | Type | (mol %) | Type | (mol %) | Type | (mol %) | Type | (mol %) | Mw | Mw/Mn |
| Synthesis | A-1 | M-1 | 40 | M-4 | 60 | — | — | — | — | 5700 | 1.6 |
| Example 1 | | | | | | | | | | | |
| Synthesis | A-2 | M-1 | 40 | M-5 | 60 | — | — | — | — | 5800 | 1.6 |
| Example 2 | | | | | | | | | | | |
| Synthesis | A-3 | M-1 | 40 | M-6 | 60 | — | — | — | — | 5900 | 1.5 |
| Example 3 | | | | | | | | | | | |
| Synthesis | A-4 | M-1 | 40 | M-7 | 60 | — | — | — | — | 6000 | 1.7 |
| Example 4 | | | | | | | | | | | |
| Synthesis | A-5 | M-1/M-2 | 45/10 | M-8 | 45 | — | — | — | — | 6200 | 1.7 |
| Example 5 | | | | | | | | | | | |
| Synthesis | A-6 | M-1 | 40 | M-9 | 60 | — | — | — | — | 5500 | 1.5 |
| Example 6 | | | | | | | | | | | |
| Synthesis | A-7 | M-1/M-2 | 45/10 | M-10 | 45 | — | — | — | — | 5400 | 1.6 |
| Example 7 | | | | | | | | | | | |
| Synthesis | A-8 | M-1 | 20 | M-10/M-11 | 40/40 | — | — | — | — | 5900 | 1.4 |
| Example 8 | | | | | | | | | | | |
| Synthesis | A-9 | M-1 | 50 | M-8/M-12 | 35/15 | — | — | — | — | 5800 | 1.6 |
| Example 9 | | | | | | | | | | | |
| Synthesis | A-10 | M-1 | 60 | M-4 | 40 | — | — | — | — | 6300 | 1.5 |
| Example 10 | | | | | | | | | | | |
| Synthesis | A-11 | M-1 | 30 | M-5 | 60 | — | — | M-13 | 10 | 6900 | 1.7 |
| Example 11 | | | | | | | | | | | |
| Synthesis | A-12 | M-17/M-3 | 30/10 | M-4 | 60 | — | — | — | — | 6700 | 1.7 |
| Example 12 | | | | | | | | | | | |
| Synthesis | A-13 | M-17 | 35 | M-4 | 55 | M-14 | 10 | — | — | 6800 | 1.7 |
| Example 13 | | | | | | | | | | | |
| Synthesis | A-14 | M-17 | 35 | M-5 | 55 | M-15 | 10 | — | — | 6500 | 1.7 |
| Example 14 | | | | | | | | | | | |
| Synthesis | A-15 | M-17 | 35 | M-9 | 55 | M-16 | 10 | — | — | 6600 | 1.7 |
| Example 15 | | | | | | | | | | | |

### Radiation-Sensitive Acid Generator [B] and Other Radiation-Sensitive Acid Generators [C]

Compounds represented by the following (B-1) to (B-13) and (C-1) to (C-11) were used as radiation-sensitive acid generators.

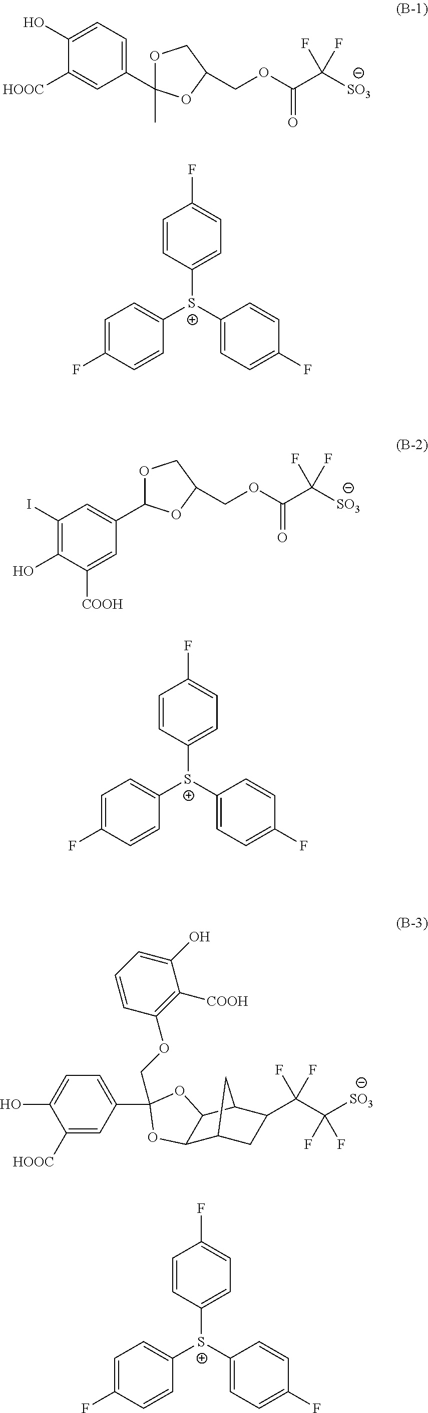

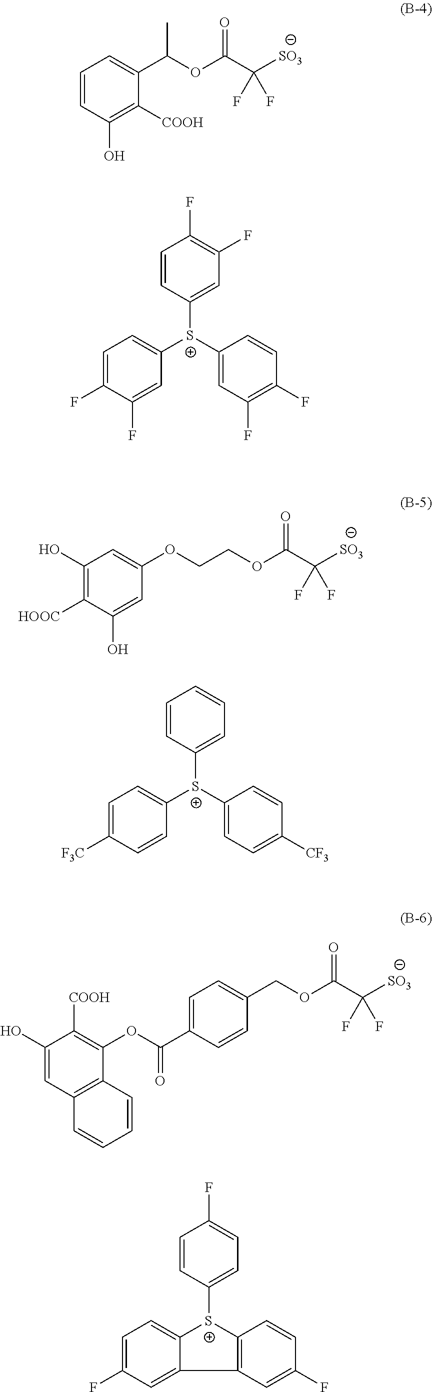

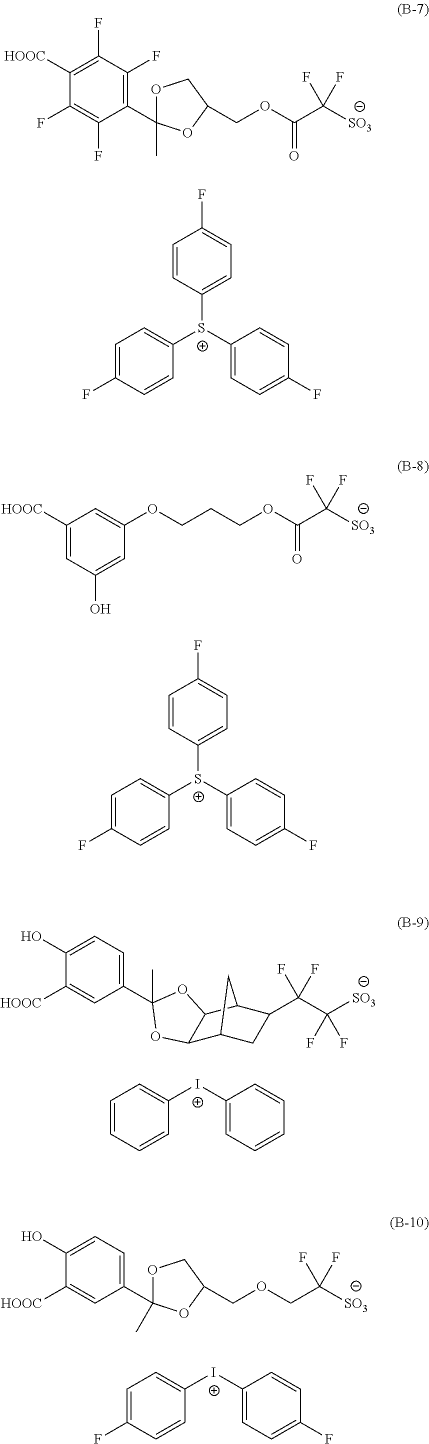

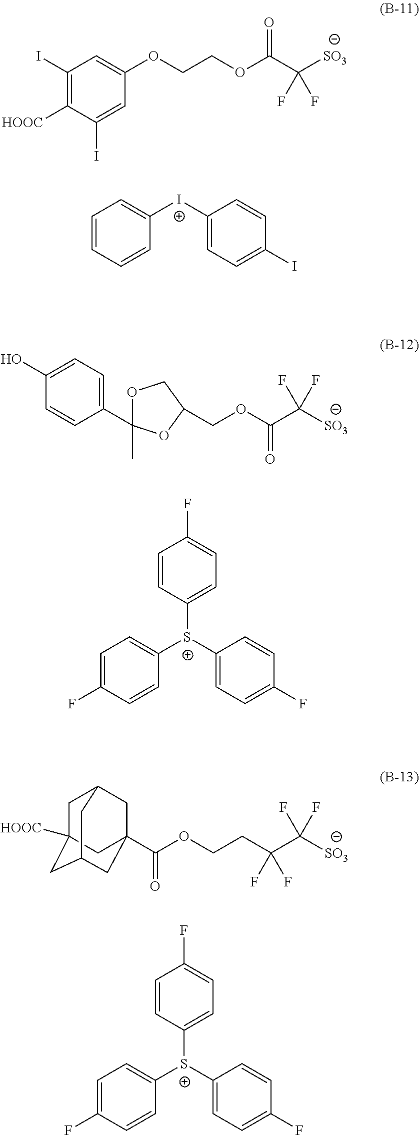

### <Acid Diffusion Controlling Agent [D]>

Compounds represented by the following (D-1) to (D-4) were used as acid diffusion controlling agents.

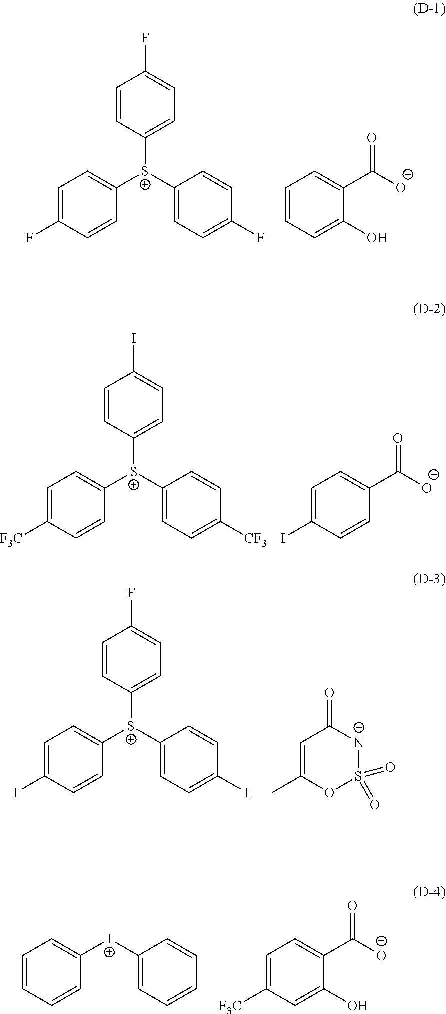

### <Solvent [E]>

E-1: Propylene glycol 1-monomethyl ether
E-2: Propylene glycol monomethyl ether acetate
E-3: Methyl 2-hydroxyisobutyrate
E-4: Diacetone alcohol

### Example 1

Blended and mixed were 100 parts by mass of [A] the polymer (A-1), 10 parts by mass of [B] the radiation-sensitive acid generator (B-1), 10 parts by mass of [C] the radiation-sensitive acid generator (C-1), 20 mol % of [D] the acid diffusion controlling agent (D-1) with respect to the total of (B-1) and (C-1), 2,000 parts by mass of [E] the solvent (E-1), and 4,800 parts by mass of (E-2), followed by filtration through a filter having a pore size of 0.20 μm to prepare a radiation-sensitive composition (R-1).

### Examples 2 to 43 and Comparative Examples 1 to 5

Radiation-sensitive compositions (R-2) to (R-43) and (CR-1) to (CR-5) were prepared in the same manner as in Example 1 except that the types and the blending amounts of respective components shown in the following Table 2 were used.

TABLE 2

| | | | | Radiation- | | Other radiation- | | | | | |
|---|---|---|---|---|---|---|---|---|---|---|---|
| | | | | sensitive acid | | sensitive acid | | Acid diffusion | | | |
| | | Polymer [A] | | generator [B] | | generators [C] | | controlling | | | |
| | Radiation- | | Content | | Content | | Content | agent [D] | | Solvent [E] | |
| | sensitive | | (parts by | | (parts by | | (parts by | | Content | | Content |
| | composition | Type | mass) | Type | mass) | Type | mass) | Type | (mol %) | Type | (parts by mass) |
| Example 1 | R-1 | A-1 | 100 | B-1 | 10 | C-1 | 10 | D-1 | 20 | E-1/E-2 | 2,000/4800 |
| Example 2 | R-2 | A-2 | 100 | B-1 | 10 | C-1 | 10 | D-1 | 20 | E-1/E-2 | 2,000/4800 |
| Example 3 | R-3 | A-3 | 100 | B-1 | 10 | C-1 | 10 | D-1 | 20 | E-1/E-2 | 2,000/4800 |
| Example 4 | R-4 | A-4 | 100 | B-1 | 10 | C-1 | 10 | D-1 | 20 | E-1/E-2 | 2,000/4800 |
| Example 5 | R-5 | A-5 | 100 | B-1 | 10 | C-1 | 10 | D-1 | 20 | E-1/E-2 | 2,000/4800 |
| Example 6 | R-6 | A-6 | 100 | B-1 | 10 | C-1 | 10 | D-1 | 20 | E-1/E-2 | 2,000/4800 |
| Example 7 | R-7 | A-7 | 100 | B-1 | 10 | C-1 | 10 | D-1 | 20 | E-1/E-2 | 2,000/4800 |
| Example 8 | R-8 | A-8 | 100 | B-1 | 10 | C-1 | 10 | D-1 | 20 | E-1/E-2 | 2,000/4800 |
| Example 9 | R-9 | A-9 | 100 | B-1 | 10 | C-1 | 10 | D-1 | 20 | E-1/E-2 | 2,000/4800 |
| Example 10 | R-10 | A-10 | 100 | B-1 | 10 | C-1 | 10 | D-1 | 20 | E-1/E-2 | 2,000/4800 |
| Example 11 | R-11 | A-11 | 100 | B-1 | 10 | C-1 | 10 | D-1 | 20 | E-1/E-2 | 2,000/4800 |
| Example 12 | R-12 | A-12 | 100 | B-1 | 10 | C-1 | 10 | D-1 | 20 | E-1/E-2 | 2,000/4800 |
| Example 13 | R-13 | A-1 | 100 | B-2 | 10 | C-1 | 10 | D-1 | 20 | E-1/E-2 | 2,000/4800 |
| Example 14 | R-14 | A-1 | 100 | B-3 | 10 | C-1 | 10 | D-1 | 20 | E-1/E-2 | 2,000/4800 |
| Example 15 | R-15 | A-1 | 100 | B-4 | 10 | C-1 | 10 | D-1 | 20 | E-1/E-2 | 2,000/4800 |
| Example 16 | R-16 | A-1 | 100 | B-5 | 10 | C-1 | 10 | D-1 | 20 | E-1/E-2 | 2,000/4800 |
| Example 17 | R-17 | A-1 | 100 | B-6 | 10 | C-1 | 10 | D-1 | 20 | E-1/E-2 | 2,000/4800 |
| Example 18 | R-18 | A-1 | 100 | B-7 | 10 | C-1 | 10 | D-1 | 20 | E-1/E-2 | 2,000/4800 |
| Example 19 | R-19 | A-1 | 100 | B-8 | 10 | C-1 | 10 | D-1 | 20 | E-1/E-2 | 2,000/4800 |
| Example 20 | R-20 | A-1 | 100 | B-9 | 10 | C-1 | 10 | D-1 | 20 | E-1/E-2 | 2,000/4800 |
| Example 21 | R-21 | A-1 | 100 | B-10 | 10 | C-1 | 10 | D-1 | 20 | E-1/E-2 | 2,000/4800 |
| Example 22 | R-22 | A-1 | 100 | B-11 | 10 | C-1 | 10 | D-1 | 20 | E-1/E-2 | 2,000/4800 |
| Example 23 | R-23 | A-1 | 100 | B-1 | 10 | C-2 | 10 | D-1 | 20 | E-1/E-2 | 2,000/4800 |
| Example 24 | R-24 | A-1 | 100 | B-1 | 10 | C-3 | 10 | D-1 | 20 | E-1/E-2 | 2,000/4800 |
| Example 25 | R-25 | A-1 | 100 | B-1 | 10 | C-4 | 10 | D-1 | 20 | E-1/E-2 | 2,000/4800 |
| Example 26 | R-26 | A-1 | 100 | B-1 | 10 | C-5 | 10 | D-1 | 20 | E-1/E-2 | 2,000/4800 |
| Example 27 | R-27 | A-1 | 100 | B-1 | 10 | C-6 | 10 | D-1 | 20 | E-1/E-2 | 2,000/4800 |
| Example 28 | R-28 | A-1 | 100 | B-1 | 10 | C-7 | 10 | D-1 | 20 | E-1/E-2 | 2,000/4800 |
| Example 29 | R-29 | A-1 | 100 | B-1 | 10 | C-8 | 10 | D-1 | 20 | E-1/E-2 | 2,000/4800 |
| Example 30 | R-30 | A-1 | 100 | B-1 | 10 | C-9 | 10 | D-1 | 20 | E-1/E-2 | 2,000/4800 |
| Example 31 | R-31 | A-1 | 100 | B-1 | 10 | C-11 | 10 | D-1 | 20 | E-1/E-2 | 2,000/4800 |
| Example 32 | R-32 | A-1 | 100 | B-1 | 15 | C-1 | 5 | D-1 | 20 | E-1/E-2 | 2,000/4800 |
| Example 33 | R-33 | A-1 | 100 | B-1 | 5 | C-1 | 15 | D-1 | 20 | E-1/E-2 | 2,000/4800 |
| Example 34 | R-34 | A-1 | 100 | B-1 | 10 | C-1 | 10 | D-2 | 20 | E-1/E-2 | 2,000/4800 |
| Example 35 | R-35 | A-1 | 100 | B-1 | 10 | C-1 | 10 | D-3 | 20 | E-1/E-2 | 2,000/4800 |
| Example 36 | R-36 | A-1 | 100 | B-1 | 10 | C-1 | 10 | D-4 | 20 | E-1/E-2 | 2,000/4800 |
| Example 37 | R-37 | A-1 | 100 | B-1 | 40 | — | — | D-4 | 20 | E-1/E-2 | 2,000/4800 |
| Example 38 | R-38 | A-1 | 100 | B-1 | 10 | C-1 | 10 | D-1 | 20 | E-1/E-2/E-3 | 1,000/1,800/4,000 |
| Example 39 | R-39 | A-1 | 100 | B-1 | 10 | C-1 | 10 | D-1 | 20 | E-1/E-2/E-4 | 1,000/1,800/4,000 |
| Example 40 | R-40 | A-13 | 100 | B-1 | 10 | — | — | D-1 | 20 | E-1/E-2 | 2,000/4800 |
| Example 41 | R-41 | A-14 | 100 | B-11 | 10 | — | — | D-4 | 20 | E-1/E-2 | 2,000/4800 |
| Example 42 | R-42 | A-15 | 100 | B-11 | 10 | — | — | D-1 | 20 | E-1/E-2 | 2,000/4800 |
| Example 43 | R-43 | A-13 | 100 | B-11 | 10 | — | — | D-4 | 20 | E-1/E-2/E-3 | 1,000/1,800/4,000 |
| Comparative | CR-1 | A-1 | 100 | B-1 | 20 | — | — | D-1 | 20 | E-1/E-2 | 2,000/4800 |
| Example 1 | | | | | | | | | | | |
| Comparative | CR-2 | A-1 | 100 | — | — | C-1 | 20 | D-1 | 20 | E-1/E-2 | 2,000/4800 |
| Example 2 | | | | | | | | | | | |
| Comparative | CR-3 | A-1 | 100 | B-12 | 10 | C-1 | 10 | D-1 | 20 | E-1/E-2 | 2,000/4800 |
| Example 3 | | | | | | | | | | | |
| Comparative | CR-4 | A-1 | 100 | B-13 | 10 | C-1 | 10 | D-1 | 20 | E-1/E-2 | 2,000/4800 |
| Example 4 | | | | | | | | | | | |
| Comparative | CR-5 | A-1 | 100 | B-1 | 10 | C-10 | 10 | D-1 | 20 | E-1/E-2 | 2,000/4800 |
| Example 5 | | | | | | | | | | | |

### <Formation of Resist Pattern>

Each of the radiation-sensitive compositions prepared as described above was applied using a spin coater (CLEAN TRACK ACT12, manufactured by Tokyo Electron Ltd.) to the surface of a 12-inch silicon wafer with a 20 nm thick underlayer film (AL412, manufactured by Brewer Science) formed thereon. Soft baking (SB) was performed at 100 °C for 60 seconds, followed by cooling at 23 °C for 30 seconds, to form a resist film having a thickness of 40 nm. Then, the resist film was irradiated with EUV light using an EUV exposure machine (model “NXE3300”, manufactured by ASML, NA=0.33, lighting condition: Conventional, s=0.89). Then, the resist film was subjected to post exposure baking (PEB) at 100 °C for 60 seconds. Subsequently, development was performed at 23 °C for 30 seconds using a 2.38 wt % aqueous TMAH solution to form a positive-tone 50 nm-pitch 25 nm contact hole pattern.

### <Evaluation>

The sensitivity, the CDU, and storage stability of each of the radiation-sensitive compositions were evaluated by measuring each of the formed resist patterns in accordance with the following methods. A scanning electron microscope (“CG-5000” manufactured by Hitachi High-Technologies Corporation) was used for measuring the length of the resist patterns. The evaluation results are shown in Table 3 below.

### [Sensitivity]

An exposure amount at which the 25 nm contact hole pattern was formed in the formation of the resist pattern was defined as an optimum exposure amount, and the optimum exposure amount was defined as sensitivity (mJ/cm²). The smaller the value is, the better the sensitivity is. The sensitivity was determined as “S” (extremely good) when the value was less than 56 mJ/cm², “A” (good) when the value was 56 mJ/cm² or more and less than 58 mJ/cm², “B” (slightly good) when the value was 58 mJ/cm² or more and 61 mJ/cm² or less, and “C” (poor) when the value was more than 61 mJ/cm².

### [CDU]

The 25 nm contact hole pattern was observed from above using the scanning electron microscope, and the length thereof was measured at 800 arbitrary points in total. The dimensional variation (30) was determined and taken as CDU (nm). The smaller the value of the CDU is, the smaller the variation in hole diameter in the long period is and the better the CDU is. The CDU was determined as “S” (extremely good) in the case of less than 3.4 nm, “A” (good) in the case of 3.4 nm or more and less than 3.6 nm, “B” (slightly good) in the case of 3.6 nm or more and less than 3.8 nm, and “C” (poor) in the case of 3.8 nm or more.

### [Storage Stability]

After preparation, the radiation-sensitive composition was stored at −15 °C for 2 weeks or at 35 °C for 2 weeks. Thereafter, an optimum exposure amount for forming a 150 nm line-and-space pattern in resist pattern formation with a KrF exposure machine (“NSR-S203B” manufactured by NIKON) was determined. The rate of change of the optimum exposure amount S_{H} of the radiation-sensitive composition stored at 35 °C for 2 weeks with respect to the optimum exposure amount Si of the radiation-sensitive composition stored at −15 °C for 2 weeks was calculated based on formula. A case where the rate of change in sensitivity was 1.0% or more was judged as “C”, and a case where the rate of change in sensitivity was 0.7% or more and less than 1.0% was judged as “B”. Otherwise, the rate of change in sensitivity was determined as “A”.

Rate of change in sensitivity (%)={|S_{H}−S_{L}|/S_{L}}×100

TABLE 3

| | Radiation-sensitive | | | Storage |
|---|---|---|---|---|
| | composition | Sensitivity | CDU | stability |
| Example 1 | R-1 | A | B | A |
| Example 2 | R-2 | A | A | A |
| Example 3 | R-3 | A | B | A |
| Example 4 | R-4 | A | B | A |
| Example 5 | R-5 | A | A | A |
| Example 6 | R-6 | A | A | A |
| Example 7 | R-7 | A | A | A |
| Example 8 | R-8 | A | A | A |
| Example 9 | R-9 | A | A | A |
| Example 10 | R-10 | A | B | A |
| Example 11 | R-11 | B | B | A |
| Example 12 | R-12 | A | A | A |
| Example 13 | R-13 | A | A | A |
| Example 14 | R-14 | A | B | A |
| Example 15 | R-15 | A | B | A |
| Example 16 | R-16 | A | A | A |
| Example 17 | R-17 | B | B | A |
| Example 18 | R-18 | A | B | A |
| Example 19 | R-19 | A | A | B |
| Example 20 | R-20 | S | B | A |
| Example 21 | R-21 | S | A | A |
| Example 22 | R-22 | A | B | A |
| Example 23 | R-23 | S | A | A |
| Example 24 | R-24 | A | S | A |
| Example 25 | R-25 | B | A | A |
| Example 26 | R-26 | A | B | A |
| Example 27 | R-27 | A | A | A |
| Example 28 | R-28 | B | A | A |
| Example 29 | R-29 | A | A | A |
| Example 30 | R-30 | A | B | A |
| Example 31 | R-31 | A | A | A |
| Example 32 | R-32 | B | A | A |
| Example 33 | R-33 | A | B | B |
| Example 34 | R-34 | A | A | A |
| Example 35 | R-35 | B | A | A |
| Example 36 | R-36 | S | A | A |
| Example 37 | R-37 | A | A | A |
| Example 38 | R-38 | A | B | A |
| Example 39 | R-39 | A | B | A |
| Example 40 | R-40 | A | A | B |
| Example 41 | R-41 | A | A | B |
| Example 42 | R-42 | A | A | B |
| Example 43 | R-43 | A | A | A |
| Comparative | CR-1 | C | B | A |
| Example 1 | | | | |
| Comparative | CR-2 | A | A | C |
| Example 2 | | | | |
| Comparative | CR-3 | A | A | C |
| Example 3 | | | | |
| Comparative | CR-4 | B | A | C |
| Example 4 | | | | |
| Comparative | CR-5 | C | C | A |
| Example 5 | | | | |

As is apparent from the results in Table 3, all the radiation-sensitive compositions of Examples were better in sensitivity and CDU while securing storage stability as compared with the radiation-sensitive compositions of Comparative Examples.

According to the radiation-sensitive composition and the pattern forming method of the present disclosure, sensitivity and CDU can be improved while securing storage stability. Therefore, the radiation-sensitive composition and the resist pattern formation method can be suitably used for the formation of a fine resist pattern in a lithography process for various electronic devices such as semiconductor devices and liquid crystal devices.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising:
a polymer comprising: a structural unit (I) which comprises an acid-dissociable group; and, optionally, a structural unit (II) which comprises an acid generation structure that comprises a first organic acid anion and an iodonium cation liberated from the first organic acid anion and is capable of generating through exposure an acid which dissociates the acid-dissociable group;
a radiation-sensitive acid generator that comprises a second organic acid anion and a second onium cation liberated from the second organic acid anion and is capable of generating through exposure an acid which dissociates the acid-dissociable group;
an acid diffusion controlling agent that comprises a third organic acid anion and a third onium cation liberated from the third organic acid anion and is capable of generating through exposure an acid which does not dissociate the acid-dissociable group; and
a solvent,
wherein
the radiation-sensitive acid generator comprises a compound represented by formula (1), and
at least one of conditions (i) and (ii) is satisfied:
(i) the polymer comprises the structural unit (II), and
(ii) at least a part of the second onium cation or the third onium cation is an iodonium cation: 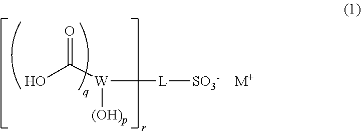
wherein in the formula (1),
W is a (p+q+1)-valent aromatic ring having 5 to 20 ring members, when there are a plurality of Ws, the plurality of Ws are identical to or different from each other,
L is a (r+1)-valent linking group,
r is an integer of 1 to 3,
p is an integer of 0 to 3, when there are a plurality of ps, the plurality of ps are identical to or different from each other,
q is an integer of 1 to 3, when there are a plurality of qs, the plurality of qs are identical to or different from each other, and
M⁺ is a monovalent onium cation.

2. The radiation-sensitive composition according to claim 1,
wherein in the formula (1),
when r is 1, p is an integer of 1 to 3; or
when r is greater than or equal to 2, at least one p is an integer of 1 to 3.

3. The radiation-sensitive composition according to claim 2, wherein at least one —OH is bonded to a carbon atom adjacent to a carbon atom to which a —COOH of the formula (1) is bonded.

4. The radiation-sensitive composition according to claim 1, wherein in the formula (1), W is a benzene ring.

5. The radiation-sensitive composition according to claim 1, wherein in the formula (1), r is 1.

6. The radiation-sensitive composition according to claim 1, wherein in the formula (1), L comprises at least one structure selected from the group consisting of a cyclic structure, an ester bond, and an ether bond.

7. The radiation-sensitive composition according to claim 1, wherein in the formula (1), a fluorine atom or a fluorinated hydrocarbon group is bonded to a carbon atom adjacent to a sulfur atom of —SO₃⁻.

8. The radiation-sensitive composition according to claim 1, wherein a content of the radiation-sensitive acid generator in the radiation-sensitive composition is 5 parts by mass or more and 60 parts by mass or less with respect to 100 parts by mass of the polymer.

9. The radiation-sensitive composition according to claim 1, wherein the iodonium cation is represented by formula (P1): 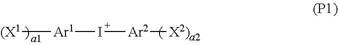
wherein in the formula (1),
Ar¹ is a (a1+1)-valent aromatic ring having 5 to 20 carbon atoms,
Ar² is a (a2+1)-valent aromatic ring having 5 to 20 carbon atoms,
X¹ and X² are each independently a monovalent organic group having 1 to 20 carbon atoms, a nitro group, a hydroxy group, or a halogen atom, when there are a plurality of X¹s and there are a plurality of X²s, the plurality of X¹s are identical to or different from each other and the plurality of X²s are identical to or different from each other, and
a1 and a2 are each independently an integer of 0 to 5.

10. The radiation-sensitive composition according to claim 9, wherein in the formula (P1), Ar¹ and Ar² are each a benzene ring.

11. The radiation-sensitive composition according to claim 1, wherein the polymer further comprises a structural unit (IV) which comprises a phenolic hydroxy group.

12. The radiation-sensitive composition according to claim 1, further comprising a high fluorine-containing polymer that is higher in mass content of fluorine atoms than the polymer.

13. A pattern formation method, comprising:
directly or indirectly applying the radiation-sensitive composition according to claim 1 onto a substrate to form a resist film;
exposing the resist film to light; and
developing the exposed resist film with a developer.

14. The pattern formation method according to claim 13, wherein exposing comprises exposing the resist film to an extreme ultraviolet ray or an electron beam.
